(19) United States
(12) Patent Application Publication
(10) Pub. No.: US 2003/0139352 A1
(43) Pub. Date: Jul. 24, 2003

(54) Inhibitors of ABC drug transporters in cancer cells

(76) Inventor: Grant L. Schoenhard, San Carlos, CA (US)

Correspondence Address: Janet M. McNicholas, Ph.D., McAndrews, Held & Malloy, Ltd., 34th Floor, 500 West Madison Street, Chicago, IL, 60661, US

(21) Appl. No.: 10/159,012

(22) Filed: May 30, 2002

Related U.S. Application Data
(63) Continuation-in-part of application No. 10/003,215, filed on Oct. 30, 2001.

(51) Int. Cl. A61K 31/7048; A61K 31/704; A61K 31/485; A61K 31/4745; A61K 31/337; A61K 31/65; A61K 31/17; A61K 31/28

(52) U.S. Cl. 514/27; 514/34; 514/251; 514/282; 514/283; 514/449; 514/183; 514/492; 514/588; 514/152

(57) ABSTRACT

The present invention relates to cancer, including in cancer and, in particular, to opioid compounds that are inhibitors of drug transporters of the ABC protein superfamily. The invention relates to methods of treating cancer using anti-tumor agents and opioid inhibitors of such transporters. The invention also relates to methods for selecting or designing compounds for the ability to inhibit drug transporter proteins and to methods of inhibiting drug transporter proteins. The invention concerns the new use of opioid receptor antagonists in the treatment of a cancer patient who has developed a resistance to a therapeutically active substance.

### CROSS-REFERENCE TO RELATED APPLICATIONS

This application is a continuation-in-part of co-pending U.S. patent application Ser. No. 10/003,215, filed Oct. 30, 2001, hereby incorporated by reference.

### BACKGROUND

ATP-binding cassette (ABC) proteins play a central role in living cells through their role in nutrient uptake, protein, drug and antibiotic secretion, osmoregulation, antigen presentation, signal transduction and others. The majority of ABC proteins have a translocation function either in import of substrates or secretion of cellular products or xenobiotics.

The ATP binding cassette (ABC) superfamily is one of the largest superfamilies known. With the multiplication of genome sequencing projects, new sequences appear every week in the GenBank database. Members of this family posses a highly conserved protein or module, the ABC module, that displays the WalkerA and WalkerB motifs separated by a short, highly conserved, sequence (consensus LSGGQ (SEQ ID NO: 1) called a signature sequence or linker peptide. Most ABC cassette proteins are primary transporters for movement of molecules across biological membranes. The substrates handled by these transporters are extraordinarily varied ranging from small molecules to macromolecules.

ABC proteins of particular interest are the drug transporters associated with multidrug resistance in humans. The human multidrug resistance protein family is composed of a number of well characterized members (See, e.g., Borst et al, J. Natl. Cancer Inst. 92:1295-1302 (2000)). Originally implicated in the resistance of tumor cells to chemotherapeutic agents, the multi-drug resistance protein P-glycoprotein (PGP), a product of the MDR1 gene, belongs to the ATP-binding cassette family of proteins. P-glycoprotein is an ATP-dependent drug transporter that is predominantly found in the apical membranes of a number of epithelial cell types in the body, including the luminal membrane of the brain capillary endothelial cells that make up the blood-brain barrier. PGP is expressed in the human intestine, blood brain barrier, liver, and other tissues. (See, e.g., Matheny et al., Pharmacology 21(7): 778-796 (2001); Nolnar et al., Anticancer Res. 17(1A): 481-486 (1997)). Expression of PGP, localized to cell membranes may affect the bioavailability of drug molecules that are substrates for this transporter. Drugs that inhibit P-glycoprotein can alter the absorption, disposition and elimination of co-administered drugs and can enhance bioavailability or cause unwanted drug-drug interactions. Interaction with PGP can be studied using either direct assays of drug transport in polarized cell systems or with indirect assays such as drug-stimulated ATPase activity and inhibition of the transport of fluorescent substrates.

ABC cassette proteins have been implicated in the resistance of many human cancers to traditional chemotherapeutic agents, i.e., multidrug resistance. The major documented cause of multidrug resistance of cancers is the overexpression of P-glycoprotein, which is capable of pumping structurally diverse anti-tumor drugs from cells. See D. Houseman et al., A Molecular Genetic Approach to the Problem of Drug Resistance in Chemotherapy, 504-517 (1987) (Academic Press, Inc.); R. Fine and B. Chabner, Multidrug Resistance, in Cancer Chemotherapy 8, 117-128 (H. Pinedo and B. Chabner eds. 1986); Ann Rev. Biochem 58:137-171 (1989). Increased expression of the gene encoding P-glycoprotein is found in many malignant cells, including leukemias, lymphomas, sarcomas and carcinomas, and may be upregulated by the onset of a malignancy and/or cellular contact with chemotherapeutic agents. Once active, P-glycoprotein is believed to function as a “hydrophobic vacuum cleaner” which expels hydrophobic drugs from targeted cells. Such drugs include many anti-cancer drugs and cytotoxic agents, such as vinca alkaloids, anthracyclines, epipodophyllotoxins, taxanes, actinomycins, colchicine, puromycin, toxic peptides (e.g., valinomycin), topotecan, and ethidium bromide. See I. Pastan and M. Gottesman, New England J. Med. 1388, 1389 Table 1 (May 28, 1987).

Tumor cells expressing elevated levels of the multiple drug transporter accumulate far less anti-tumor agents intracellularly than tumor cells having low levels of this transporter. The degree of resistance of certain tumor cells has been documented to correlate with both elevated expression of the drug transporter and reduced accumulation of anti-tumor drugs. See M. Gottesman and I. Pastan, J. Biol. Chem. 263, 12163 (1988); see also A. Fojo et al., Cancer Res. 45: 3002 (1985).

The multidrug resistance proteins, MRP1 and MRP2, have been implicated in mediating the ATP-dependent transport conjugates with glutathione, glucuronic acid, or sulfate. Both transporters have been associated with multiple drug resistance of malignant tumors because of their capacity to transport drug conjugates and drug complexes across the plasma membrane into the extracellular fluids, thus reducing the concentration of the drugs and therefore limiting their potential for the treatment of tumors. Some differences in substrate specificity have been observed between the substrate specificity of MRP1 and MRP2 and P-glycoprotein. MRP1 and MRP2 may be termed conjugate-transporting proteins functioning in detoxification and, because of their role in glutathione disulfide export, in the defense against oxidative stress.

Reduced intracellular levels of anti-tumor agents in the tumor suppresses chemotherapeutic efficacy. Tumors having elevated levels of the multiple drug transporter require therapeutic doses of cancer suppressants far in excess of tumors exhibiting lower levels of drug transporters. Agents that inhibit the active efflux of anti-tumor agents by the drug transporter or agents that potentiate the efficacy of chemotherapeutic agents would enhance the activity of various anti-tumor agents on tumor cells. Inhibition of PGP function in PGP-mediated multidrug resistance has been shown to lead to a net accumulation of anti-cancer agent in the cells. For example, verapamil a known calcium channel blocker was shown to sensitize MDR cells to vinca alkaloids in vitro and in vivo.

ABC drug transporters found in normal host cells mediate transport of numerous compounds in the duodenum, the liver, the kidneys, the brain, and putatively in other tissues. These host transporters have the capacity to transport drugs, drug conjugates and drug complexes across plasma membranes into extracellular fluids or back into associated tissues.

The ability of the drug transporter proteins such as ABC proteins to actively transport therapeutic substances from cells, including both tumor and host cells, has impeded the development of therapies for a wide variety of disorders and conditions in multicellular hosts, particularly in humans. Thus, a continuing need exists for methods to increase the ability of clinicians to administer bioactive substances across biological membranes.

### SUMMARY OF THE INVENTION

The present invention provides methods and compositions with anti-tumor agents and opioid inhibitors of ABC drug transporters. Exemplary opioid inhibitors useful in such methods and compositions are nalmefene, nalthrexone and naloxone. Additionally, opioid inhibitors of the invention are described having a pharmacophore as defined herein. Opioid inhibitors of the invention are also described as having a structure of a formula as defined herein.

The present invention provides methods of increasing the efficacy of an anti-tumor agent by co-administering to a subject suffering from a cancer (e.g., a cancer patient or host) a dose, including a therapeutic or sub-therapeutic dose, of an anti-tumor agent and a dose of an opioid inhibitor of an ABC drug transporter. For example, the dose of the opioid inhibitor of the ABC drug transporter is sufficient to reduce efflux of the anti-tumor agent from a cancer cell, to increase the intracellular concentration of the anti-tumor agent in a cancer cell, and/or to inhibit a drug transporter of a subject. Also, for example, the dose of an opioid inhibitor of the ABC transporter facilitates the distribution of anti-tumor agents into tissues and.or cells of a subject where in the absence of the inhibitor the uninhibited ABC transporter facilitated efflux is so high as to prevent attainment of therapeutic concentrations of anti-tumor agents in those tissues and/or cells.

The invention provides methods of decreasing toxicity associated with treating a cancer patient by co-administering a dose, including a therapeutic or a sub-therapeutic dose, of an anti-tumor agent and a dose of an opioid inhibitor of an ABC drug transporter. For example, the anti-tumor agent is a substrate of an ABC drug transporter and the dose of opioid inhibitor is sufficient to reduce efflux of the anti-tumor agent from a cancer cell, to increase the concentration of the anti-tumor agent within the cancer cell, and/or to inhibit a host drug transporter. Also, for example, the dose of an opioid inhibitor of the ABC transporter facilitates the distribution of anti-tumor agents into tissues and/or cells of a subject where in the absence of the inhibitor the uninhibited ABC transporter facilitated efflux is so high as to prevent attainment of therapeutic concentrations of anti-tumor agents in those tissues and/or cells.

The invention also provides compositions for treating cancer cells, including multidrug resistant cancer cells, with a combination of an anti-tumor agent and an opioid inhibitor of a ABC drug transporter. For example, the anti-tumor agent is a substrate of the ABC drug transporter and the dose of opioid inhibitor is sufficient to reduce efflux of the anti-tumor agent from a cancer cell, to increase the concentration of the anti-tumor agent within the cancer cell, and/or to inhibit a host drug transporter. Also, for example, the dose of an opioid inhibitor of the ABC transporter facilitates the distribution of anti-tumor agents into tissues and/or cells of a subject where in the absence of the inhibitor the uninhibited ABC transporter facilitated efflux is so high as to prevent attainment of therapeutic concentrations of anti-tumor agents in those tissues and/or cells.

The invention provides an opioid inhibitor of the ABC drug transporters that has a pharmacophore defined by a hydrogen bonding moiety at a three-dimensional location corresponding to the hydroxyl at position 3 of naltrexone, a hydrogen bonding moiety at a three-dimensional location corresponding to the hydroxyl at position 14 of naltrexone, a hydrophobic moiety at a three-dimensional location corresponding to the cyclopropyl moiety appended to the nitrogen of naltrexone, and a region of electron density at a three-dimensional location corresponding to the ethylene moiety at 6-position of naltrexone. Additionally, the invention provides ABC drug transporter opioid inhibitors of the formula:

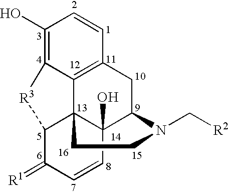

wherein R¹ is CH₂ or O;

wherein R² is a cycloalkyl, unsubstituted aromatic, alkyl or alkenyl; and

wherein R³ is O, CH₂ or NH.

Exemplary opioid inhibitors of ABC drug transporters are nalmefene, naltrexone and naloxone.

The invention also provides methods of enhancing the anti-tumor activity of an anti-tumor agent against a cancer cell by contacting the cancer cell with the anti-tumor agent and an opioid inhibitor of an ABC drug transporter in an amount effective to inhibit a drug transporter in the cancer cell. For example, the cancer cell expresses an ABC drug transporter and the anti-tumor agent is a substrate of the ABC drug transporter.

The invention also provides methods of suppressing growth of a cancer cell, including a multidrug resistant cancer cell, expressing an ABC drug transporter protein by contacting the cancer cell with an amount, including a therapeutic or a sub-therapeutic amount, of an anti-tumor agent in the presence of an opioid inhibitor of the ABC drug transporter.

The invention also provide methods of inhibiting a P-glycoprotein in a subject suffering from cancer. For example, a P-glycoprotein inhibiting amount of nalmefene, naltrexone or naloxone is administered to the subject before, with, or after the administration to the subject of a therapeutic or sub-therapeutic amount of an anti-tumor agent.

The invention also provides methods of identifying compounds, including anti-tumor agents, for improved treatment of cancer. The method includes identifying an anti-tumor agent, assaying the ability of the anti-tumor agent to be transported across a membrane by an ABC protein, and repeating the transport assay to determine whether addition of an opioid inhibitor of an ABC drug transporter inhibits transport of the anti-tumor agent across the membrane. The desired compound is identified as a compound that is transported by an ABC protein and whose ABC protein-mediated transport is inhibited by an opioid inhibitor. The desired compound inhibits the ABC transporter in various human tissues and/or cells that prevent the attainment of therapeutic concentrations of anti-tumor agents in those human tissues and/or cells. The invention provides such anti-tumor compounds.

The invention also provides methods for screening for an opioid inhibitor of an ABC drug transporter by determining whether a potential opioid inhibitor inhibits growth of a cancer cell in the presence of an amount, including a therapeutic or a sub-therapeutic amount, of an anti-tumor agent. Inhibition of growth is assayed by comparing the growth of a cancer cell which expresses the ABC drug transporter, with growth of a second cancer cell which does not produce the ABC drug transporter. Both are grown in the presence of an amount, including a therapeutic or a sub-therapeutic amount, of the anti-tumor agent.

The invention also provides methods for screening for an opioid inhibitor of an ABC drug transporter. The method includes contacting a potential opioid inhibitor of an ABC drug transporter protein with the ABC drug transporter protein in the presence of a compound that is a known opioid inhibitor, including, for example, nalmefene, naltrexone or naloxone, wherein the compound is detectable by any means of detection and measuring the amount of detected compound bound to the ABC drug transporter. The measured amount is compared to the amount of detected compound bound by the ABC drug transporter when the drug transporter is contacted with the compound alone. An ABC drug transporter inhibitor is identified by a decreased amount of compound bound to the ABC drug transporter when the potential inhibitor is present. The compound may be detectably labeled (e.g., radio labeled) or detected by spectroscopy (e.g., U. V., mass spectral, infrared, flame ionization, electrochemical) or other detectors capable of quantifying compounds alone or in tandem with chromatography.

The invention also provides methods of treating cancer in a subject (e.g., an animal host, including a human) by administering an anti-tumor agent and an amount of an opioid inhibitor of an ABC drug transporter, including, for example, nalmefene, naltrexone or naloxone, sufficient to increase the intracellular concentration of the anti-tumor agent. The ABC drug transporter inhibitor increases the susceptibility of the cancer cell to the anti-tumor agent. An amount of such opioid inhibitors also or alternative inhibits the ABC transporters in various tissues and cells of the subject that prevents the attainment of therapeutic concentrations of anti-cancer agents in those tissues and cells.

### BRIEF DESCRIPTION OF THE DRAWINGS

FIG. 1 illustrates the chemical structures of nalmefene, naltrexone, naloxone, 6-β-naltrexol and nalorphine.

FIG. 2 presents an overlay of the opioid analogues, nalmefene, naltrexone, naloxone, 6-β-naltrexol and nalorphine.

FIG. 3A shows the molecular orbitals and electrostatic potential of nalmefene as calculated using Spartan (Wavefunction, Inc.).

FIG. 3B shows the molecular orbitals and electrostatic potential of naloxone as calculated using Spartan (Wavefunction, Inc.).

FIGS. 4A-4AH provide information about the 200 nearest neighbors to the opioid analogues examined in the QSAR analysis.

### DETAILED DESCRIPTION

The present invention is based in part on surprising results from transport studies that compounds previously identified as opioid receptor antagonists are inhibitors of ABC drug transporter proteins, such as the exemplary P-glycoprotein, PGP-1a. Opioid receptor antagonists, including, for example, naltrexone, have been clinically used for decades but their transport characteristics have never been evaluated using contemporary cultured cell lines technology. Administration of opioid receptor antagonists, such as nalmefene, naltrexone or naloxone, unexpectedly results in increased intracellular concentrations of co-administered therapeutic agents in cells expressing an ABC drug transporter protein, particularly in multidrug resistant cancer cells expressing PGP1a. The present invention provides a novel class of drug transporter inhibitors that act by inhibiting ABC transporter proteins and/or their associated ATPase as described herein and further provides a pharmacophore that identifies new drug targets that are inhibitors of ABC transporter proteins.

As used herein, the terms “transporter” and “drug transporter” refer to a protein for the carrier-mediated influx and efflux of drugs and endocytosis of biologically active molecules across a cell membrane barrier, including across a gut, liver, or blood-brain barrier. An inhibitor of a transporter is expected to increase the efficacy of an active agent according to the invention, wherein the transporter inhibitor reduces efflux across the cellular membrane of a cancer cell, increases influx into the cancer cell, and/or inhibits a host drug transporter. Preferably the drug transporter protein is a member of the ABC superfamily, referred to as an “ABC drug transporter.” The ABC drug transporter may either be a multidrug resistance protein (MDR) or a multidrug resistance-associated protein (MRP).

Among the ABC superfamily of drug transporters, there are several closely conserved regions, the nucleotide binding motifs of the WalkerA region and WalkerB region, and the short consensus sequence (leucine-serine-glycine-glycine-glutamine, or LSGGQ, SEQ ID NO: 1). ABC drug transporters generally contain the consensus sequence or a very closely related sequence. The QSAR analysis of the present invention provides the very surprising result that the opioid receptor antagonists that act as ABC drug transporter inhibitors are expected to bind in the region of this LSGGQ consensus sequence, as well as additional binding regions. Thus, the present invention defines a strictly conserved inhibition site shared among all ABC drug transporter proteins. Therefore, the ABC drug transporter inhibitor, including compounds identified as opioid receptor antagonists, according to the present invention will function as an inhibitor of a ABC drug transporter protein that shares the LSGGQ conserved sequence.

Thus, the present invention is based up the identification of a new class of drug transporter inhibitors. The term “drug transporter inhibitor” or “ABC drug transporter inhibitor” refers to a compound that binds to an ABC drug transporter protein and inhibits, e.g., either completely blocks or merely slows transport of compounds across biological barriers. Compounds, such as drugs, that inhibit drug transporters can alter the absorption, disposition and elimination of co-administered drugs and can enhance bioavailability or cause unwanted drug-drug interactions. Interaction with drug transporters can be studied using either direct assays of drug transport in polarized cell systems and/or with indirect assays such as drug-stimulated ATPase activity or inhibition of the transport of fluorescent substrates. Drugs affected by the drug transporter, P-glycoprotein, include ondasetron, dexamethasone, domperidone, loperamide, doxorubicin, neifinavir, indinevir, sugguinavir, erythromycin, digoxin, vinblastine, paclitaxel, invermectin and cyclosporin. Known inhibitors of P-glycoprotein include ketoconazole, verapamil, quinidine, cyclosporin, digoxin, erythromycin and loperamide. The present invention unexpectedly identifies opioid receptor antagonists, such as nalmefene, naltrexone and naloxone, as potent inhibitors of the drug transporter, P-glycoprotein. The QSAR analysis of the invention demonstrates that the opioid receptor antagonists are also inhibitors of ABC drug transporters, especially of homologues of human PGP1a. The present invention provides opioid inhibitors of the ABC transporters that have a pharmacophore defined by a hydrogen bonding moiety at a three-dimensional location corresponding to the hydroxyl at position 3 of naltrexone, a hydrogen bonding moiety at a three-dimensional location corresponding to the hydroxyl at position 14 of naltrexone, a hydrophobic moiety at a three-dimensional location corresponding to the cyclopropyl moiety appended to the nitrogen of naltrexone, and a region of electron density at a three-dimensional location corresponding to the ethylene moiety at 6-position of naltrexone.

An “opioid receptor antagonist” is an opioid compound or composition including any active metabolite of such compound or composition, that in a sufficient amount attenuates (e.g., blocks, inhibits, prevents or competes with) the action of an opioid receptor agonist. An opioid receptor antagonist binds to and blocks (e.g., inhibits) opioid receptors on nociceptive neurons. Opioid receptor antagonists include: nalmefene, naltrexone (e.g., marketed in 50 mg dosage forms as ReVia® or Trexan®), naloxone (e.g., marketed as Narcan®), methylnaltrexone, methiodide, nalorphine, naloxonazine, nalide, nalmexone, nalbuphine, nalorphine dinicotinate, naltrindole (NTI), naltrindole isothiocyanate (NTII), naltriben (NTB), nor-binaltorphimine (nor-BNI), β-funaltrexamine (β-FNA), BNTX, cyprodime, ICI-174,864, LY117413, MR2266, or an opioid receptor antagonist having the same pentacyclic nucleus as nalmefene, naltrexone, nalorphine, nalbuphine, thebaine, levallorphan, oxymorphone, butorphanol, buprenorphine, levorphanol meptazinol, pentazocine, dezocine, or their pharmacologically effective esters or salts. In some preferred embodiments, the opioid receptor antagonist is nalmefene, naltrexone, naloxone, or mixtures thereof.

The term “opioid” refers to compounds which bind to specific opioid receptors and have agonist (e.g., activation) or antagonist (e.g., inactivation) effects at these receptors, and thus are “opioid receptor agonists” or “opioid receptor antagonists,” respectively.

In particular, the present invention contemplates enhancing the efficacy of anti-tumor agents by co-administering the anti-tumor agent with an ABC transporter inhibitor such as an opioid receptor antagonist. The opioid receptor antagonists, nalmefene, naltrexone and naloxone, are particularly suited for the compositions and methods of the present invention. Although some inhibitors of ABC drug transporters are known in the art, many of these are toxic, especially if used repeatedly over a period of time. For example, when used orally, ketoconazole has been associated with hepatic toxicity, including some fatalities. The opioid receptor antagonists, however, have limited side effects, and particularly at the low concentrations administered in the present invention. Each of the opioid receptor antagonists nalmefene, naltrexone and naloxone have been administered for human use in antagonistically effective amounts for treatment of opioid overdose and addictions.

Co-administration of an opioid ABC drug transporter inhibitor and an anti-tumor agent is expected to provide more effective treatment of cancer. Concurrent administration of the two agents may provide greater therapeutic effects in vivo than the anti-tumor agent provides when administered singly. For example, concurrent administration may permit a reduction in the dosage of the anti-tumor agent with achievement of a similar therapeutic effect. Alternatively, the concurrent administration may produce a more rapid or complete anti-tumor effect than could be achieved with the anti-tumor agent alone.

“Co-administer,” “co-administration,” “concurrent administration” or “co-treatment” refers to administration of an anti-tumor agent and an opioid drug transporter inhibitor, in conjunction or combination, together, or before or after each other. The anti-tumor agent and the opioid drug transporter inhibitor may be administered by different routes. For example, the anti-tumor agent may be administered orally and the opioid drug transporter inhibitor intravenously, or vice versa. The anti-tumor agent and the opioid drug transporter inhibitor are preferably both administered orally, as immediate or sustained release formulations. The anti-tumor agent and opioid drug transporter inhibitor may be administered simultaneously or sequentially, as long as they are given in a manner to allow both agents to achieve effective concentrations to yield their desired therapeutic effects.

“Therapeutic effect” or “therapeutically effective” refers to an effect or effectiveness that is desirable and that is an intended effect associated with the administration of an active agent according to the invention. A “therapeutic amount” is the amount of an active agent sufficient to provide a therapeutic effect. “Sub-therapeutic amount” is an amount of the active agent which does not cause a therapeutic effect in a subject administered the active agent alone, but when used in combination with an opioid drug transporter inhibitor is therapeutically effective.

Therapeutic effectiveness is based on a successful clinical outcome, and does not require that the anti-tumor agent or agents kill 100% of the cancer cells. Success depends on achieving a level of anti-tumor activity at the site of the cancer that is sufficient to inhibit the cancer cells in a manner that tips the balance in favor of the host. When host defenses are maximally effective, the anti-tumor effect required may be minimal.

The term “drug resistance” refers to the circumstance when a disease does not respond to a treatment drug. Drug resistance can be either intrinsic or acquired. “Multidrug resistance” means a specific type of drug resistance characterized by cross-resistance of a disease to more than one functionally and/or structurally unrelated drugs. The term “ABC transporter-mediated multidrug resistance” refers to multidrug resistance due to the activity of an ABC drug transporter protein.

One of the major problems of cancer chemotherapy is the existence of drug resistance in tumors resulting in reduced responsiveness to chemotherapy. Some human cancers, e.g., kidney and colon carcinoma, are drug resistant before treatment begins, while in others drug resistance develops over successive rounds of chemotherapy. One type of drug resistance, called multidrug resistance, is characterized by cross resistance to functionally and structurally unrelated drugs. Typical drugs that are affected by the multidrug resistance are doxorubicin, vincristine, vinblastine, coichicine and actinomycin D, and others. At least some multidrug resistance is a complex phenotype which has been linked to a high expression of a cell membrane drug efflux transporter called MDR1 protein, also known as P-glycoprotein. This membrane “pump” has broad specificity and acts to remove from the cell a wide variety of chemically unrelated toxins. See e.g., Endicott, J. A., et al. “The Biochemistry of P-Glycoprotein-Mediated Multidrug Resistance”, Ann. Rev. Biochem. Vol. 58, pgs. 127-71, 1989.

Cancer chemotherapy with cytotoxic agents can be successful only if the tumor cells are more sensitive than normal cells, whose destruction is incompatible with survival of the host. Success, defined either as cure or clinically significant remission, is not readily explained by the still popular idea that tumor cells are more susceptible to cytotoxic agents because they are dividing more rapidly than vital normal cells, e.g. hematopoietic precursor cells. That rapid proliferation does not wholly account for the selective drug sensitivity of tumors is demonstrated by the common observations that some drug-sensitive cancers are not rapidly dividing, and that many rapidly proliferating tumors exhibit resistance. To say that the mechanisms accounting for the success or failure of chemotherapy for most human tumors is incompletely understood today is undoubtedly an understatement.

However, recent evidence suggests that the selectivity of chemotherapy for the relatively few tumors ever cured by drugs depends, to a large extent, upon their easy susceptibility to undergo apoptosis, i.e. to kill themselves. Many cytotoxic drugs that kill cells by crippling cellular metabolism at high concentration can trigger apoptosis in susceptible cells at much lower concentration. This appears to account for the unusual chemosensitivity of many lymphoid tumors, since many normal lymphocytes are “primed” to undergo self destruction as an essential part of the mechanism for generating and controlling diversity of the immune response. Increased susceptibility to apoptosis may also be acquired by tumor cells as a byproduct of the genetic changes responsible for malignant transformation. For example, tumor cells with constitutive c-myc expression may undergo apoptosis in response to DNA damage by anticancer agents, whereas normal cells are able to pause at checkpoints in the cell cycle to repair the damage, or may not be cycling at all, rendering them highly resistant to apoptosis in this setting.

Anti-tumor agents from a number of classes of compounds can be co-administered with an opioid inhibitor of an ABC drug transporter protein. Preferably, the anti-tumor agent is selected from the following classes of compounds: Alkylating Agents, such as nitrogen mustards, ethyleneimines, methylamelamines, alkyl sulfonates, nitrosoureas, or triazene, Antimetabolites, such as folic acid analogs, pyrimidine analogs, purine analogs, Vinca alkaloids, taxanes, epipodophyllotoxins, Anthracyclines, Antiproliferative agents, Tubulin Binding agents, Enediynes, anthracededione, substituted urea, methylhydrazine derivatives, the Pteridine family of drugs, Taxanes, Dolastatins, Topoiosomerase inhibitors, Mytansinoids, and Platinum coordination complexes.

The anti-tumor agent is advantageously selected from the following compounds or their active metabolites, or derivatives or analogs thereof: Doxorubicin, Daunorubicin, Vinblastine, Vincristine, Calicheamicin, Etoposide, Etoposide phosphate, CC-1065, Duocarmycin, KW-2189, Methotrexate, Methopterin, Aminopterin, Dichloromethotrexate, Docetaxel, Paclitaxel, Epithiolone, Combretastatin, Combretastatin A4 Phosphate, Dolastatin 10, Dolastatin 11, Dolastatin 15, Topotecan, Camptothecin, Mitomycin C, Porfiromycin, 5-Fluorouracil, 6-Mercaptopurine, Fludarabine, Tamoxifen, Cytosine arabinoside, Adenosine Arabinoside, Colchicine, Carboplatin, Mitomycin C, Bleomycin, Melphalan, Cyclosporin A, Chloroquine, Maytansine, Gleevec (imantinib mesylate) or Cisplatin. By derivative is intended a compound that results from reacting the named compound with another chemical moiety, and includes a pharmaceutically acceptable salt, acid, base or ester of the named compound. By analog is intended a compound having similar structural and functional properties, such as biological activities, to the named compound.

For administration to human subjects or in the treatment of any clinical conditions, the pharmaceutical compositions, doses, or dosage forms of this invention may be utilized in compositions such as capsules, tablets or pills for oral administration, suppositories for rectal administration, liquid compositions for parenteral administration and the like. One or more doses may be administered according to methods of the invention.

The pharmaceutical compositions, doses, or dosage forms of this invention may be used in the form of a pharmaceutical preparation, for example, in solid or semisolid form, which contains one or more of the drug transporter inhibitors, as an active ingredient, alone, or in combination with one or more therapeutic agents. Any drug transporter inhibitor or therapeutic agent, according to the invention, may be in admixture with an organic or inorganic carrier or excipient suitable for external, enteral or parenteral applications. A drug transporter inhibitor may be compounded, for example, with the usual non-toxic, pharmaceutically acceptable carriers for capsules, tablets, pellets, suppositories, and any other form suitable for use. The carriers which can be used are water, glucose, lactose, gum acacia, gelatin, mannitol, starch paste, magnesium, trisilicate, talc, corn starch, keratin, colloidal silica, potato starch, urea and other carriers suitable for use in manufacturing preparations, in solid or semisolid form, and in addition auxiliary, stabilizing, thickening and coloring agents and perfumes may be used. A drug transporter inhibitor, alone or in conjunction with a therapeutic agent, is included in a pharmaceutical composition dose, or dosage form in an amount sufficient to produce the desired effect upon the process or condition, including a variety of conditions and diseases in humans.

For preparing solid compositions such as tablets, a drug transporter inhibitor, alone or in conjunction with therapeutic agent, is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that a drug transporter inhibitor, alone or in conjunction with therapeutic agent, is dispersed evenly throughout the composition so that the composition may be readily subdivided into equally effective unit dosage forms such as capsules, tablets, caplets, or pills. The capsules, tablets, caplets, or pills of the novel pharmaceutical composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Controlled release (e.g., slow-release or sustained-release) dosage forms, as well as immediate release dosage forms are specifically contemplated according to the present invention.

Compositions in liquid forms in which a therapeutic agent may be incorporated for administration orally or by injection include aqueous solution, suitable flavored syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably, compositions are administered by the oral or nasal respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug transporter inhibitor alone, or in combination with a therapeutic agent, may be administered to the human subject by known procedures including but not limited to oral, sublingual, intramuscular, subcutaneous, intravenous, intratracheal, transmucosal, or transdermal modes of administration. When a combination of these compounds are administered, they may be administered together in the same composition, or may be administered in separate compositions. If a therapeutic agent and a drug transporter inhibitor are administered in separate compositions, they may be administered by similar or different modes of administration, or may be administered simultaneously with one another, or shortly before or after the other.

Drug transporter inhibitors alone, or in combination with therapeutic agents are formulated in compositions with a pharmaceutically acceptable carrier (“pharmaceutical compositions”). The carrier must be “acceptable” in the sense of being compatible with the other ingredients of the formulation and not deleterious to the recipient thereof. Examples of suitable pharmaceutical carriers include lactose, sucrose, starch, talc, magnesium stearate, crystalline cellulose, methyl cellulose, carboxymethyl cellulose, glycerin, sodium alginate, gum arabic, powders, saline, water, among others. The formulations may conveniently be presented in unit dosage and may be prepared by methods well-known in the pharmaceutical art, by bringing an active compound into association with a carrier or diluent, or optionally with one or more accessory ingredients, e.g., buffers, flavoring agents, surface active agents, or the like. The choice of carrier will depend upon the route of administration. Pharmaceutical compositions may be administered as solid or semisolid formulations, including as capsules, tablets, caplets, pills or patches. Formulations may be presented as an immediate release or as a controlled release (e.g., slow release or sustained release) formulation.

For oral or sublingual administration, a formulation may be presented as capsules, tablets, caplets, powders, granules or a suspension, with conventional additives such as lactose, mannitol, corn starch or potato starch; with binders such as crystalline cellulose, cellulose derivatives, acacia, corn starch, gelatins, natural sugars such as glucose or beta-lactose, corn sweeteners, natural and synthetic gums such as acacia, tragacanth, or sodium alginate, carboxymethylcellulose, polyethylene glycol, waxes, or the like; with disintegrators such as corn starch, potato starch, methyl cellulose, agar, bentonite, xanthan gums, sodium carboxymethyl-cellulose or the like; or with lubricants such as talc, sodium oleate, sodium stearate, magnesium stearate, sodium benzoate, sodium acetate, sodium chloride or the like.

For transdermal administration, compounds may be combined with skin penetration enhancers such as propylene glycol, polyethylene glycol, isopropanol, ethanol, oleic acid, N-methylpyrrolidone, or the like, which increase the permeability of the skin to the compounds, and permit the compounds to penetrate through the skin and into the bloodstream. Compound/enhancer compositions also may be combined additionally with a polymeric substance such as ethylcellulose, hydroxypropyl cellulose, ethylene/vinylacetate, polyvinyl pyrrolidone, or the like, to provide the composition in gel form, which can be dissolved in solvent such as methylene chloride, evaporated to the desired viscosity, and then applied to backing material to provide a patch.

For intravenous, intramuscular, or subcutaneous administration, compounds may combined with a sterile aqueous solution which is preferably isotonic with the blood of the recipient. Such formulations may be prepared by dissolving solid active ingredient in water containing physiologically compatible substances such as sodium chloride, glycine, or the like, and/or having a buffered pH compatible with physiological conditions to produce an aqueous solution, and/or rendering said solution sterile. Formulations may be present in unit or multi-dose containers such as sealed ampoules or vials.

When a drug transporter inhibitor is used in combination with a therapeutic agent, the amount of the therapeutic agent administered may be a therapeutic or sub-therapeutic amount. As used herein, a “therapeutic” amount is the amount of a therapeutic agent which causes a therapeutic effect in a subject administered the therapeutic agent alone. The amount of the drug transporter inhibitor may be an amount sufficient to reduce efflux of the anti-tumor agent from a cancer cell, to increase the intracellular concentration of the anti-tumor agent in a cancer cell, to inhibit a host drug transporter. Also, the amount of an opioid inhibitor of the ABC transporter may be an amount that facilitates the distribution of anti-tumor agents into tissues and/or cells of a subject where, in the absence of the inhibitor, the uninhibited ABC transporter facilitated efflux is so high as to prevent attainment of therapeutic concentrations of anti-tumor agents in those tissues and/or cells. The amount of the drug transporter inhibitor additionally may be an amount effective to enhance the therapeutic potency of and/or attenuate the adverse side effects of the therapeutic agent. The optimum amounts of a drug transporter inhibitor administered alone or in combination with a therapeutic agent will of course depend upon the particular drug transporter inhibitor and therapeutic agent used, the carrier chosen, the route of administration, and/or the pharmacokinetic properties of the subject being treated.

The present invention is described in the following examples which are set forth to aid in the understanding of the invention, and should not be construed to limit in any way the invention as defined in the claims which follow thereafter.

### EXAMPLE 1

### Assays of Human Pgp-Mediated Transport with Opioid Receptor Antagonists

Porcine kidney-derived, LLC-PK₁ (American Type Culture Collection, Manassas, Va., A.T.C.C. CL-101), cells expressing human PGP cDNA (designated 15B-J) were cultured in 24 well Falcon™ culture inserts at 37° C. on an orbital shaker. Transport assays were conducted in 24 well Falcon™ culture inserts with Hanks Balanced Salt Solution (HBSS) buffered with the addition of 10 mM HEPES (pH 7.2).

The test substances, nalmefene, naltrexone and naloxone, were purchased from Sigma-Aldrich (St. Louis, Mo.). Stock solutions of the compounds were made in DMSO, and dilutions of these in transport buffer were prepared for assay in the monolayers. The DMSO concentration (0.55%) was constant for all conditions within the experiment. All test substance and control drug solutions prepared in HBSS/HEPES buffer contained 0.55% DMSO.

The test substance was added to the donor and receiver chambers. Duplicate monolayers and thirteen nominal test substance concentrations of 0.0001, 0.0003, 0.001, 0.003, 0.01, 0.03, 0.1, 0.3, 1.0, 3.0, 10, 30 and 100 μM were used. PGP substrate [³H]-digoxin, at 5 μM was added to the donor chamber (either the apical or basolateral chamber depending on the direction of transport). After an incubation time of 90 minutes, a sample from the receiver chamber was analyzed for the amount of digoxin present. The positive control for inhibition was 25 μM ketoconazole added to donor and receiver chambers with 5 μM [³H]-digoxin added to the donor chamber. The negative control for inhibition was 5 μM [³H]-digoxin added to the donor chamber (either the apical or basolateral chamber depending on the direction of transport) with Hanks Balanced Salt Solution (HBSS) buffered with the addition of 10 mM HEPES (pH 7.2) and DMSO at 0.55% in the receiver chamber.

The rate of digoxin transported from the apical chamber to the basolateral chamber (A to B) and from the basolateral chamber to the apical chamber (B to A) was measured and apparent permeability Pₐₚₚ constants calculated. The polarization ratio P_{app B to A}/P_{app A} was calculated. A lower polarization ratio in the 15B-J cells with test substance relative to that without test substance provides evidence for inhibition of PGP-mediated digoxin transport by the test substance.

Transport of 5 μM [3H]-digoxin was measured following coincubation with the test substances at nominal concentrations in the range of 0 to 100 μM. Inhibition of digoxin transport was calculated by comparison of the digoxin polarization ratio in the presence of the test substance, to the ratio in the absence of test substance. The positive control for inhibition was 25 μM ketoconazole coincubated with digoxin. The inhibition of PGP-mediated transport in human PGP-expressing porcine kidney cell monolayers by naloxone is summarized in Table 1.

TABLE 1
Naloxone inhibition of PGP-mediated transport

| | | | | Ketoconazole |
|---|---|---|---|---|
| | | | | Normalized % |
| Naloxone | | Polarization | % Inhibition | Inhibition of |
| Concentration (μm) | | Ratio | of Digoxin | Digoxin |
| Nominal | Measured | (B→A/A→B) | Transport | Transport |
| 0 | — | 3.7 | — | — |
| 0.0001 | 0.000021 | 3.5 | 4.4 | 6.2 |
| 0.0003 | 0.000138 | 3.5 | 6.0 | 8.4 |
| 0.001 | 0.00085 | 3.4 | 7.3 | 10 |
| 0.03 | 0.0021 | 3.6 | 4.0 | 5.7 |
| 0.01 | 0.0083 | 3.8 | −3.2 | −4.5 |
| 0.03 | 0.021 | 3.5 | 4.1 | 5.7 |
| 0.1 | 0.074 | 3.8 | −1.9 | −2.7 |
| 0.3 | 0.264 | 3.3 | 11.9 | 17 |
| 1.0 | 1.04 | 3.5 | 5.5 | 7.8 |
| 3.0 | 2.79 | 4.0 | −7.7 | −11 |
| 10 | 10.0 | 3.7 | 0.3 | 0.4 |
| 30 | 31.8 | 2.6 | 29 | 41 |
| 100 | 99.8 | 1.1 | 26 | 37 |

The inhibition of PGP-mediated transport in human PGP-expressing porcine kidney cell monolayers by naltrexone is summarized in Table 2.

TABLE 2
Naltrexone inhibition of PGP-mediated transport

| | | | Ketoconazole |
|---|---|---|---|
| | | | Normalized % |
| Naltrexone | Polarization | % Inhibition | Inhibition of |
| Concentration | ratio | of Digoxin | Digoxin |
| (μM) | (B→A/A→B) | Transport | Transport |
| 0 | 4.0 | — | — |
| 0.0001 | 3.6 | 10 | 13 |
| 0.0003 | 3.5 | 14 | 19 |
| 0.001 | 3.6 | 10 | 13 |
| 0.003 | 3.7 | 8 | 11 |
| 0.01 | 3.5 | 11 | 15 |
| 0.03 | 3.8 | 5 | 6.7 |
| 0.1 | 3.5 | 14 | 19 |
| 0.3 | 3.3 | 18 | 24 |
| 1.0 | 3.4 | 14 | 19 |
| 3.0 | 3.5 | 14 | 19 |
| 10 | 3.4 | 15 | 20 |
| 30 | 3.3 | 17 | 23 |
| 100 | 2.7 | 33 | 44 |

The inhibition of PGP-mediated transport in human PGP-expressing porcine kidney cell monolayers by nalmefene is summarized in Table 3.

TABLE 3
Nalmefene inhibition of PGP-mediated transport

| | | | Ketoconazole |
|---|---|---|---|
| | | | Normalized % |
| Naltrexone | Polarization | % Inhibition | Inhibition of |
| Concentration | ratio | of Digoxin | Digoxin |
| (μM) | (B→A/A→B) | Transport | Transport |
| 0 | 4.5 | — | — |
| 0.0001 | 4.3 | 5.2 | 7.5 |
| 0.0003 | 4.2 | 7.2 | 10 |
| 0.001 | 4.4 | 2.8 | 4.0 |
| 0.003 | 4.3 | 5.1 | 7.4 |
| 0.01 | 4.3 | 3.9 | 7.4 |
| 0.03 | 4.8 | −7.2 | −10 |
| 0.1 | 4.5 | −0.3 | — |
| 0.3 | 4.8 | −5.6 | −10 |
| 1.0 | 4.6 | −2.6 | −3.2 |
| 3.0 | 4.0 | 10 | 14 |
| 10 | 3.9 | 13 | 19 |
| 30 | 2.9 | 35 | 51 |
| 100 | 2.6 | 42 | 61 |

Nalmefene, naltrexone and naloxone exhibited inhibitory behavior from about 0.1 μM to about 100 μM. Digoxin transport appears to have been inhibited at naloxone and naltrexone concentrations below 30 μM, however, the inhibition was not concentration-dependent. Digoxin transport was increasingly inhibited in response to increasing concentration of nalmefene at concentrations between 3 and 100 μM. The positive control, 25 μM ketoconazole, inhibited digoxin transport within the accepted range, indicating that the cell model performed as expected.

### EXAMPLE 2

### Assays of Human PGP-Mediated Transport with 6-B-Naltrexol

Porcine kidney-derived, LLC-PK₁, cells expressing human PGP cDNA (designated 15B-J) were cultured in 24 well Falcon™ culture inserts at 37° C. on an orbital shaker. Transport assays were conducted in 24 well Falcon™ culture inserts with Hanks Balanced Salt Solution (HBSS) buffered with the addition of 10 mM HEPES (pH 7.2).

The test substance, 6-β-naltrexol, was provided by LC Resources, Inc. (Walnut Creek, Calif.), Stock solutions of the compounds were made in DMSO, and dilutions of these in transport buffer were prepared for assay in the monolayers. The DMSO concentration (0.55%) was constant for all conditions within the experiment. All test substance and control drug solutions prepared in HBSS/HEPES buffer contained 0.55% DMSO.

The test substance was added to the donor and receiver chambers. Duplicate monolayers and thirteen nominal test substance concentrations of 0.0001, 0.0003, 0.001, 0.003, 0.01, 0.03, 0.1, 0.3, 1, 3, 10, 30 and 100 μM, were used. PGP substrate [³H]-digoxin, at 5 μM was added to the donor chamber (either the apical or basolateral chamber depending on the direction of transport). After an incubation time of 90 minutes, a sample from the receiver chamber was analyzed for the amount of digoxin present. The positive control for inhibition was 25 μM ketoconazole added to donor and receiver chambers with 5 μM [³H]-digoxin added to the donor chamber. The negative control for inhibition was 5 μM [³H]-digoxin added to the donor chamber (either the apical or basolateral chamber depending on the direction of transport) and Hanks Balanced Salt Solution (HBSS) buffered with the addition of 10 mM HEPES (pH 7.2) and DMSO at 0.55% in the receiver chamber.

Transport of 5 μM [³H]-digoxin was measured following coincubation with test substance 6-β-naltrexol, at nominal concentrations in the range of 0 to 100 μM. Inhibition of digoxin transport was calculated by comparison of the digoxin polarization ratio in the presence of the test substance, to the ratio in the absence of test substance. The positive control for inhibition was 25 μM ketoconazole coincubated with digoxin. Transport was slightly inhibited (mean of 8.5+/−7.1%) by 6-β-naltrexol in the concentration range of 0.0001 to 30 μM (Table 4). The inhibition did not appear to be concentration-dependent. At 100 μM 6-β-naltrexol, however, digoxin transport was more strongly inhibited (28%). The positive control, 25 μM ketoconazole, inhibited digoxin transport within the accepted range, indicating that the cell model performed as expected.

TABLE 4
6-β-Naltrexol Inhibition of PGP-Mediated Transport

| | | | Ketoconazole |
|---|---|---|---|
| | | | Normalized % |
| 6-β-Naltrexol | Polarization | % Inhibition | Inhibition of |
| Nominal | Ratio | of Digoxin | Digoxin |
| Concentration | (B→A/A→B) | Transport | Transport |
| 0 | 4.7 | — | — |
| 0.0001 | 4.4 | 6.4 | 8.1 |
| 0.0003 | 4.7 | 0 | — |
| 0.001 | 4.8 | −2.1 | −2.6 |
| 0.003 | 4.7 | 0 | — |
| 0.01 | 4.6 | 2.1 | 2.6 |
| 0.03 | 4.2 | 11 | 14 |
| 0.1 | 3.8 | 19 | 24 |
| 0.3 | 4.3 | 9 | 11 |
| 1.0 | 4.0 | 15 | 19 |
| 3.0 | 4.2 | 11 | 14 |
| 10 | 4.0 | 15 | 19 |
| 30 | 4.0 | 15 | 19 |
| 100 | 3.4 | 28 | 35 |
| 25 μM Ketoconazole | 1.0 | 79 | — |

The test substance 6-β-naltrexol was an inhibitor of PGP-mediated digoxin transport, in the concentration range tested.

### EXAMPLE 3

### Assays of PGP-Associated ATPase Activity with Opioid Receptor Antagonists

The test substances, nalmefene, naltrexone and naloxone, were purchased from Sigma-Aldrich. Stock solutions of the compounds were made in DMSO, and dilutions of these in TRIS-MES buffer were prepared for PGP-associated ATPase assay in the human PGP membranes (BD Biosciences, Franklin Lakes, N.J., Gentest Cat. K228). The DMSO concentration (1.0%) was constant for all conditions within the experiment.

The test substances were incubated in the PGP membranes and supplemented with MgATP, with and without sodium orthovanadate present. Orthovanadate inhibits PGP by trapping MgADP in the nucleotide binding site. Thus, the ATPase activity measured in the presence of orthovanadate represents non-PGP ATPase activity and was subtracted from the activity generated without orthovanadate to yield ATPase activity.

ATPase assays were conducted in 96-well microtiter plates. A 0.06 mL reaction mixture containing 40 μg PGP membranes, test substance, and 4 mM MgATP, in buffer containing 50 mM Tris-MES, 2 mM EGTA, 50 mM KCl, 2 mM dithiothreitol, and 5 mM sodium azide, plus organic solvent was incubated at 37° C. for 20 minutes. Triplicate incubations of ten test substance concentrations, (of 0.001 or 0.003, 0.01, 0.03, 0.1, 0.3, 1.0, 3.0, 10, 30 and 100 μM) and the test vehicle without drug, were used. Identical reaction mixtures containing 100 μM sodium orthovanadate were assayed in parallel. The reactions were stopped by the addition of 30 μl of 10% SDS+Antifoam A. The incubations were followed with addition of 200 μl of 35 mM Ammonium Molybdate in 15 mM Zinc Acetate: 10% Ascorbic Acid (1:4) and incubated for an additional 20 minutes at 37° C. Additionally, 0.06 mL aliquots of potassium phosphate standards prepared in the buffer described above, were incubated in the plates containing the test and control substances, with SDS and detection reagent added. The liberation of inorganic phosphate was detected by its absorbance at 800 nm and quantitated by comparing the absorbance to a phosphate standard curve. The concentration dependence of the PGP was analyzed for evidence of saturation of PGP-associated ATPase activity, and apparent kinetic parameters were calculated by non-linear regression. The positive control for stimulation of ATPase activity was 20 μM verapamil. Ketoconazole at 25 μM and 100 μM (co-incubated with 20 μM verapamil) was tested as a possible inhibitor of verapamil-stimulated ATPase activity.

In a semi-quantitative assay for ATPase inhibition, nalmefene, naltrexone and naloxone were shown not to limit the ATPase activity associated with PGP1a (Table 5).

TABLE 5
ATPase Activity

| Concentration | Activity (nmol/mg min) | | |
|---|---|---|---|
| (μM) | Naloxone | Naltrexone | Nalmefene |
| 100 | 1.8 | 4.6 | 3.3 |
| 30 | 1.9 | −2.0 | 2.3 |
| 10 | 2 | 2.0 | −0.5 |
| 3.0 | 1.7 | 2.1 | 0.2 |
| 1.0 | 0.4 | −0.8 | −1.6 |
| 0.3 | −0.6 | −1.4 | 1.9 |
| 0.1 | 3.6 | −0.7 | 1.1 |
| 0.03 | 0.7 | −1.6 | 1.2 |
| 0.01 | −0.8 | 0.0 | 1.3 |
| 0.003 | n.d. | −0.3 | 2.4 |
| 0.001 | −3.8 | n.d. | n.d. |
| 0.0 | 0.0 | 0.0 | 0.0 |
| 20 μM Verapamil + | 13.4* | 41.7** | n.d. |
| ketoconazole | | | |

*100 μM ketoconazole
**25 μM ketoconazole

### EXAMPLE 4

### Taxol

A. Assays for Transport of Taxol by Human PGP

Porcine kidney-derived, LLC-PK₁ (American Type Culture Collection, Manassas, Va., A.T.C.C. #CL-101), cells expressing human PGP cDNA (designated 15B-J) and the untransfected control cell line were cultured in 24 well Falcon™ culture inserts at 37° C. on an orbital shaker. Non-transfected cells (designated CLA) of the same Porcine kidney-derived, LLC-PK₁ cell line were also used. Transport assays were conducted in 24 well Falcon™ culture inserts with Hanks Balanced Salt Solution (HBSS) buffered with the addition of 10 mM HEPES (pH 7.2).

The test substance, taxol, was provided by Sigma-Aldrich (St. Louis, Mo.). A stock solution of the compound was made in DMSO, and diluted to 10 μM in transport buffer for assay in the monolayers.

Taxol at 10 μM was added to the donor chamber (either the apical or basolateral chamber depending on the direction of transport). A sample from the receiver chamber was analyzed for the amount of taxol present, after incubation times of 30, 60, 90 and 120 minutes.

TABLE 6
PGP-mediated transport of Taxol

| | | CLA (control) Monolayer | | 15B-J (PGP) Monolayer | |
|---|---|---|---|---|---|
| | | | Polarization | | Polarization |
| Minutes | Transport | pmoles in | Ratio | pmoles in | Ratio |
| Incubated | Direction | Receiver | (B→ A/A→ B) | Receiver | (B→ A/A→ B) |
| 30 | A→ B | 8.4 | 1.1 | 6 | 3.8 |
| | B→ A | 9.2 | | 24 | |
| 60 | A→ B | 13 | 1.4 | 22 | 2.6 |
| | B→ A | 18 | | 56 | |
| 90 | A→ B | 18 | 1.6 | 22 | 4.4 |
| | B→ A | 28 | | 94 | |
| 120 | A→ B | 20 | 1.9 | 24 | 5.7 |
| | B→ A | 39 | | 135 | |

Taxol was significantly transported by human PGP.

B. Assays of PGP-Associated ATPase Activity

Taxol was incubated in the human PGP membranes (BD Biosciences, Franklin Lakes, N.J., Gentest Cat. K228) and supplemented with MgATP, with and without sodium orthovanadate present. ATPase assays were conducted in 96-well microtiter plates. A 0.06 mL reaction mixture containing 40 μg PGP membranes, taxol, and 4 mM MgATP, in buffer containing 50 mM Tris-MES, 2 mM EGTA, 50 mM KCl, 2 mM dithiothreitol, and 5 mM sodium azide, plus organic solvent (1% DMSO) was incubated at 37° C. for 20 minutes. Triplicate incubations of taxol at μM concentrations shown below and the test vehicle without drug, were used. Identical reaction mixtures containing 100 μM sodium orthovanadate were assayed in parallel. The reactions were stopped by the addition of 30 μl of 10% SDS+Antifoam A. The incubations were followed with addition of 200 μl of 35 mM Ammonium Molybdate in 15 mM Zinc Acetate: 10% Ascorbic Acid (1:4) and incubated for an additional 20 minutes at 37° C. Additionally, 0.06 mL aliquots of potassium phosphate standards prepared in the buffer described above, were incubated in the plates containing the test and control substances, with SDS and detection reagent added. The liberation of inorganic phosphate was detected by its absorbance at 800 nm and quantitated by comparing the absorbance to a phosphate standard curve. The concentration dependence of the PGP was analyzed for evidence of saturation of PGP-associated ATPase activity. The positive control for stimulation of ATPase activity was 20 μM verapamil.

In a semi-quantitative assay for ATPase inhibition, taxol stimulates PGP-associated ATPase activity in a dose dependent manner. This is consistent with taxol being effectively transported by human PGP.

TABLE 7
ATPase Activity of Taxol in Human PGP Membranes

| | ATPase Activity (nmol/mg min) |
|---|---|
| (μM) | basal activity subtracted |
| 6.67 | 14.4 |
| 3.33 | 12.8 |
| 1.67 | 11.1 |
| 0.83 | 5.0 |
| 0.42 | 2.5 |
| 0.21 | −1.2 |
| 0.10 | −1.6 |
| 0.05 | −0.9 |
| 0.03 | −0.9 |
| 0.01 | −0.4 |
| 0.007 | −0.3 |
| 0 | 0.0 |

### EXAMPLE 5

### Gleevec (Imantinib Mesylate) and Gleevec Metabolite

A. Assays for Transport by Human PGP

Porcine kidney-derived, LLC-PK₁, cells expressing human PGP cDNA (designated 15B-J) and the untransfected control cell line were cultured in 24 well Falcon™ culture inserts at 37° C. on an orbital shaker. Transport assays were conducted in 24 well Falcon™ culture inserts with Hanks Balanced Salt Solution (HBSS) buffered with the addition of 10 mM HEPES (pH 7.2).

The test substance, Gleevec (imantinib mesylate), was provided by LC Resources, Inc., Walnut Creek, Calif.. Since the N-desmethyl metabolite of Gleevec (Gleevec metabolite) is believed to also have anti-tumor activity, the metabolite was also tested. Stock solutions of the compounds were made in DMSO, and diluted to 10 μM in transport buffer for assay in the monolayers.

Duplicate monolayers were used. Gleevec (imantinib mesylate) or Gleevec metabolite was added to the donor chamber (either the apical or basolateral chamber depending on the direction of transport). A sample from the receiver chamber was analyzed for the amount of Gleevec (imantinib mesylate) or Gleevec metabolite present, after various incubation times (e.g., 15, 30, 60, 90 and/or 120 minutes).

TABLE 8
PGP-mediated transport of Gleevec (imantinib mesylate)

| | | CLA (control) Monolayer | | 15B-J (PGP) Monolayer | |
|---|---|---|---|---|---|
| | | | Polarization | | Polarization |
| Minutes | Transport | pmoles in | Ratio | pmoles in | Ratio |
| Incubated | Direction | Receiver | (B→ A/A→ B) | Receiver | (B→ A/A→ B) |
| 15 | A→ B | 5.86 | 1.4 | 8.8 | 2.7 |
| | B→ A | 8.15 | | 23.5 | |
| 30 | A→ B | 11.88 | 1.8 | 15.1 | 3.5 |
| | B→ A | 18.41 | | 43 | |
| 60 | A→ B | 30.3 | 2.0 | 33.4 | 3.2 |
| | B→ A | 50.1 | | 81 | |
| 90 | A→ B | 53 | 1.8 | 55 | 2.9 |
| | B→ A | 66 | | 98 | |
| 120 | A→ B | 70 | 1.8 | 65 | 3.1 |
| | B→ A | 69 | | 106 | |

Gleevec (imantinib mesylate) or its N-desmethyl metobolite was significantly transported by human PGP.

TABLE 9
PGP-mediated transport of Gleevec (Metabolite)

| | | CLA (control) Monolayer | | 15B-J (PGP) Monolayer | |
|---|---|---|---|---|---|
| | | | Polarization | | Polarization |
| Minutes | Transport | pmoles in | Ratio | pmoles in | Ratio |
| Incubated | Direction | Receiver | (B→ A/A→ B) | Receiver | (B→ A/A→ B) |
| 15 | A→ B | 8.4 | 0.1 | 22.4 | 0.4 |
| | B→ A | 1.0 | | 8.3 | |
| 30 | A→ B | 2.76 | 0.4 | 2.4 | 5.3 |
| | B→ A | 0.86 | | 18 | |
| 60 | A→ B | 1.6 | 0.7 | 2.3 | 15.3 |
| | B→ A | 0.7 | | 53 | |
| 90 | A→ B | 2.0 | 0.7 | 3.5 | N.D. |
| | B→ A | 0.9 | | | |
| 120 | A→ B | 1.7 | 1.3 | 9.2 | 11.5 |
| | B→ A | 1.9 | | 96 | |

B. Assays of PGP-Associated ATPase Activity

Gleevec (imantinib mesylate) or its N-desmethyl metabolite were incubated in the membranes and supplemented with MgATP, with and without sodium orthovanadate present. ATPase assays were conducted in 96-well microtiter plates. A 0.06 mL reaction mixture containing 40 μg PGP membranes, Gleevec (imantinib mesylate) or Gleevec metabolite, and 4 mM MgATP, in buffer containing 50 mM Tris-MES, 2 mM EGTA, 50 mM KCl, 2 mM dithiothreitol, and 5 mM sodium azide, plus organic solvent was incubated at 37° C. for 20 minutes. Triplicate incubations of gleevec concentrations of 0.1 μM, 0.2 μM, 0.4 μM, 0.8 μM, 1.6 μM, 3.13 μM, 6.25 μM, 12.5 μM, 25 μM, 50 μM and 100 μM and the test vehicle without drug, were used. Identical reaction mixtures containing 100 μM sodium orthovanadate were assayed in parallel. The reactions were stopped by the addition of 30 μl of 10% SDS+Antifoam A. The incubations were followed with addition of 200 μl of 35 mM Ammonium Molybdate in 15 mM Zinc Acetate: 10% Ascorbic Acid (1:4) and incubated for an additional 20 minutes at 37° C. Additionally, 0.06 mL aliquots of potassium phosphate standards prepared in the buffer described above, were incubated in the plates containing the test and control substances, with SDS and detection reagent added. The liberation of inorganic phosphate was detected by its absorbance at 800 nm and quantitated by comparing the absorbance to a phosphate standard curve. The concentration dependence of the PGP was analyzed for evidence of saturation of PGP-associated ATPase activity, and apparent kinetic parameters were calculated by non-linear regression. The positive control for stimulation of ATPase activity was 20 μM verapamil.

In a semi-quantitative assay for ATPase inhibition, gleevec (imantinib mesylate) was shown to stimulate PGP-specific ATPase activity (see Table 10).

TABLE 10
ATPase Activity

| | Gleevec-induced |
|---|---|
| Concentration | Activity |
| (μM) | (nmol/mg min) |
| 100 | 11.8 |
| 50 | 7.6 |
| 25 | 4.1 |
| 12.5 | 1.0 |
| 6.25 | −0.7 |
| 3.13 | −3.0 |
| 1.6 | −2.7 |
| 0.8 | −2.6 |
| 0.4 | −2.0 |
| 0.2 | −1.7 |
| 0.1 | 0.9 |
| 0.0 | 0.0 |
| 20 μM Verapamil | 21 |

In a semi-quantitative assay for ATPase inhibition, the N-desmethyl metabolite of Gleevec was also shown to stimulate PGP-specific ATPase activity (see Table 11).

TABLE 11
ATPase Activity

| | Gleevec |
|---|---|
| | Metabolite-Induced |
| Concentration | Activity |
| (μM) | (nmol/mg min) |
| 100 | 2.4 |
| 50 | 3.0 |
| 25 | 1.7 |
| 12.5 | −1.0 |
| 6.25 | −1.6 |
| 3.13 | −1.6 |
| 1.6 | −3.4 |
| 0.8 | −2.7 |
| 0.4 | −2.1 |
| 0.2 | −1.1 |
| 0.1 | −0.7 |
| 0.0 | 0.0 |
| 20 μM Verapamil | 20 |

### EXAMPLE 6

### Molecular Modeling Analysis

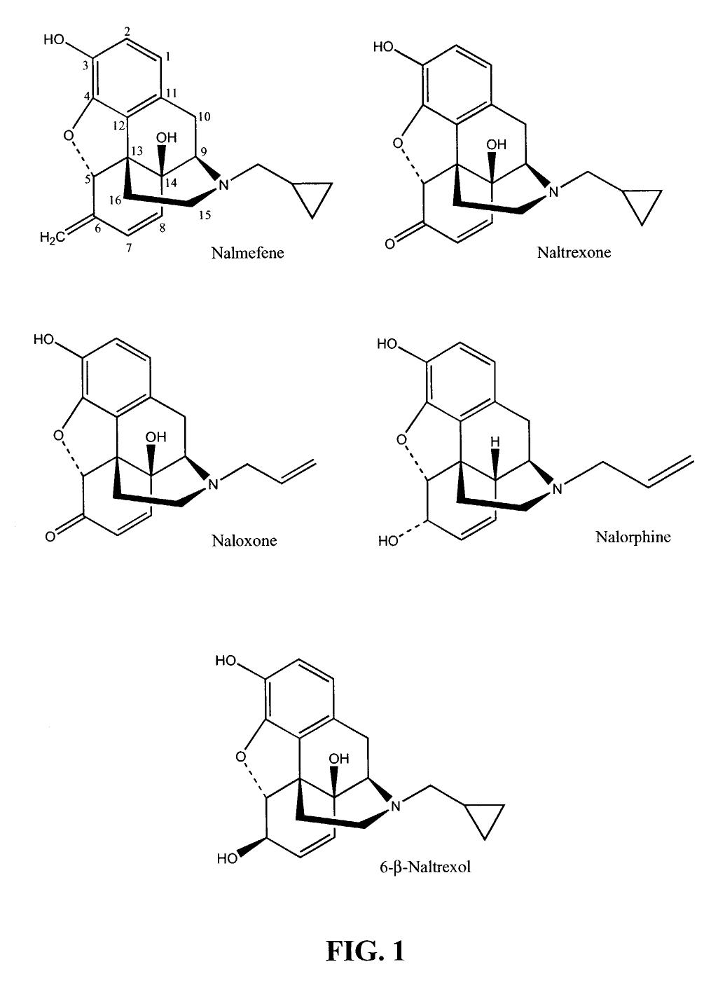

A molecular modeling analysis was performed on a series of compounds, including opioid analogues, to elucidate their mode of interaction and to determine a pharmacophore for drug transporter inhibitors useful in the present invention. Exemplary compounds in this study were nalmefene, naltrexone, naloxone, 6-β-naltrexol and nalorphine. The structures of compounds are illustrated in FIG. 1. The compounds are structurally very similar, and exhibit two measured activities. “Activity 1” is characterized by a low capacity, high affinity binding site with activity ranging from 0.3 nM to greater than 200 μM. On the other hand, “activity 2” is characterized by a high capacity, low affinity binding site with activity ranging from 10 μM to greater than 100 μM. Table 12 provides the biological activities for each of the exemplary compounds.

TABLE 12
Biological Activity of Exemplary Compounds

| Compound | Activity 1 | | Activity 2 | |
|---|---|---|---|---|
| Nalmefene | 0.3 | nM | 100 | μM |
| Naltrexone | 0.3 | nM | 100 | μM |
| Naloxone | 1.0 | nM | 30 | μM |
| 6-β-Naltrexol | 0.1 | nM | 100 | μM |

| | | |
|---|---|---|
| Nalorphine | N/A | N/A |

In performing the calculations for the molecular modeling analysis, two assumptions were made. First, nalorphine exhibits no measurable activity. Second, the structures of the compounds as represented in the Merck Index represent are the active forms of the compound.

An important difference in these compounds is that nalorphine lacks the hydroxyl group in the central ring at position 14 (see, e.g., FIG. 1), indicating that this hydroxyl group is a requirement for activity. The most active compounds (nalmefene and naltrexone) each have a hydrophobic group (cyclopropyl) tethered to the nitrogen, indicating that a hydrophobic moiety is partially responsible for the higher activity in these compounds. This moiety may be viewed as a necessary, but not sufficient condition, since several of the inactive compounds also possess this hydrophobic region. Initial activity data suggest that the electron density present at this location in naloxone (due to the ethylene substituent [C═C]) is contributory to its lower activity. The observation that 6-β-Naltrexol is even less active is attributed to the hydroxyl substituent at the 6 position being oriented β to the ring system, perhaps penetrating a sterically limited region in the transporter.

In summary, the analysis indicates that the presence of the hydroxyl group at the 14-position may be required for activity, since nalorphine, with no calculated activity, lacks this moiety. In addition, the two most active compounds (nalmefene and naltrexone) possess an ethylene group and a carbonyl group respectively at the 6-position. This may represent a requirement for electron density at this position, rather than a hydrogen-bond acceptor site, as there is only a one order of magnitude difference in activity (0.3 nM vs. 3 nM) between the ethylene group (nalmefene) and the carbonyl group (naltrexone). There is a potential steric limit for substituent size or directionality at the 6-position, based on the analysis of 6-β-Naltrexol indicates that its hydroxyl group in a direction that penetrates into this region. Finally, a hydrophobic group is required as the N-substituent for highest activity, as naloxone, with a double bond rather than the cyclopropyl group exhibits lower activity.

When the novel analysis described above is now considered in conjunction with a recent scientific article investigated the ability of a variety of peptidomimetic thrombin inhibitors to inhibit intestinal transport. Kamm et al., “Transport of peptidomimetic thrombin inhibitors with a 3-amino-phenylalanine structure: permeability and efflux mechanism in monolayers of a human intestinal cell line (Caco-2).” Pharm. Res. 18:1110-8 (2001). It is possible to utilize additional structural information from Kamm to develop a model of interaction with PGP. Kamm et al. proposed that basic and acidic residues of amidino-phenylalanine-derived thrombin inhibitors mediate affinity to intestinal efflux pumps, presumably PGP and MRP. Structural information from Kamm et al. useful in the novel QSAR analysis of the present invention is summarized below:

TABLE 13
R-groups of compounds Kamm et al.

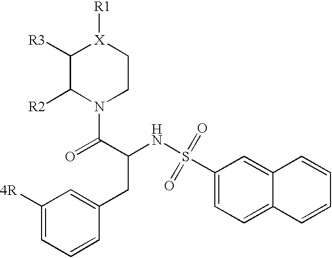

| Structure | R1 | R2 | R3 | X | R4 |
|---|---|---|---|---|---|
| 1 | Me | H | H | C | 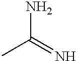 |
| 2 | H | COOH | H | C | 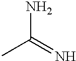 |
| 3 | H | COO-Me | H | C | 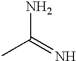 |
| 4 | H | H | COOH | C | 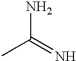 |
| 5 | H | H | COO-Me | C | 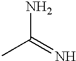 |
| 6 | COOH | H | H | C | 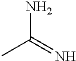 |
| 7 | COO-Me | H | H | C | 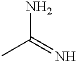 |
| 8 | COOH | H | H | C | 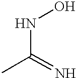 |
| 9 | COOH | H | H | C | 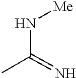 |
| 10 | H | H | H | N | 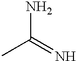 |
| 11 | 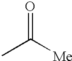 | H | H | N | 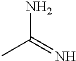 |
| (12) | Me | H | H | C | 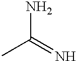 |
| 13 | Me | H | H | C | NH₂ |
| 14 | Me | H | H | C | —CH₂NH₂ |
| 15 | Me | H | H | C | 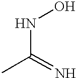 |
| 16 | Me | H | H | C | 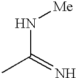 |

The intestinal permeability coefficients of the Kamm compounds were studied using Caco-2 monolayers and reverse-phase HPLC method for quantitation. Further the efflux ratios (transport from B to A:transport from A to B) were calculated. The efflux ratios for a selection of the Kamm compounds measured at 250 μM are provided in Table 14.

TABLE 14
Efflux Ratios at 250 μM

| | Efflux Ratio |
|---|---|
| Structure | B→A/A→B |
| 1 | 45.0 |
| 2 | 2.8 |
| 3 | 10.5 |
| 4 | 2.7 |
| 5 | 11.1 |
| 6 | 1.9 |
| 7 | 6.0 |
| 8 | 22.1 |
| 9 | 1.1 |
| 10 | 0.8 |
| 11 | 2.4 |

The efflux ratios the remaining Kamm compounds measured at 100 μM are provided in Table 15.

TABLE 15
Efflux Ratios at 100 μM

| | Efflux Ratio |
|---|---|
| Structure | B→A/A→B |
| 1 | 16.3 |
| 12 | 24.9 |
| 13 | 1.14 |
| 14 | 3.43 |
| 15 | 1.31 |
| 16 | 13.0 |

Comparable measurements for the opioid analogues are provided in Table 16. The data of Table 16 was obtained from the experiments described in Example 1. Efflux ratios normalized to 25 μM ketoconazole (Keto) are presented in parentheses after the measured ratios.

TABLE 16
Efflux Ratios of Opioid Analogues

| | | Hi Affinity/Low Cap | | Low Affinity/Hi Cap | |
|---|---|---|---|---|---|
| Structure | Keto @25 μM | [C] μM | B→ A/A→ B | [C] μM | B→ A/A→ B |
| Nalmefene | 1.4 | 0.0003 | 4.2 (3.0) | 100 | 2.6 (1.9) |
| Naltrexone | 1.0 | 0.0003 | 3.5 (3.5) | 100 | 2.7 (2.7) |
| Naloxone | 1.1 | 0.001 | 3.4 (3.1) | 30 | 2.6 (2.4) |
| Naloxone | | | | 100 | 2.7 (2.5) |
| 6-β-Naltrexol | 1.0 | 0.0001 | 4.4 (4.4) | 100 | 3.4 (3.4) |

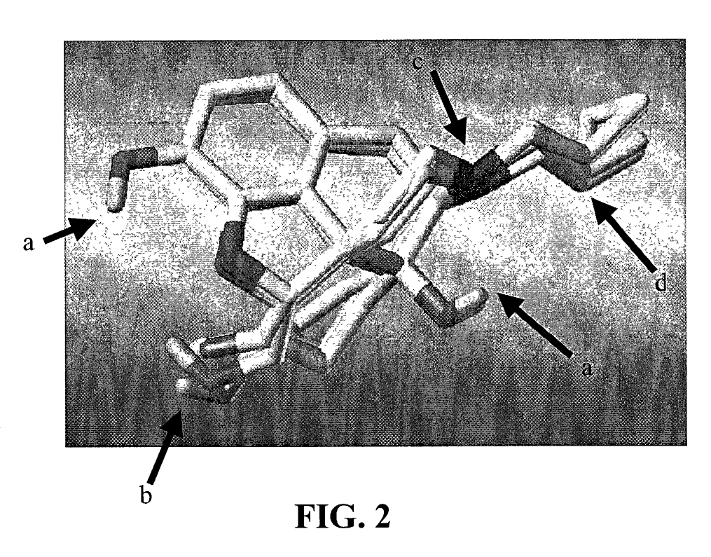

An overlay of the opioid analogue structures is presented in FIG. 2. All active (“Activity 1”) compounds share the following features: hydroxyl groups (a) at positions 3 and 14, a furan ring system, a hydrophobic region in ring system, a region of electron density at position 6 (b), and a cyclic tertiary nitrogen (c) with an appended hydrophobic group (d).

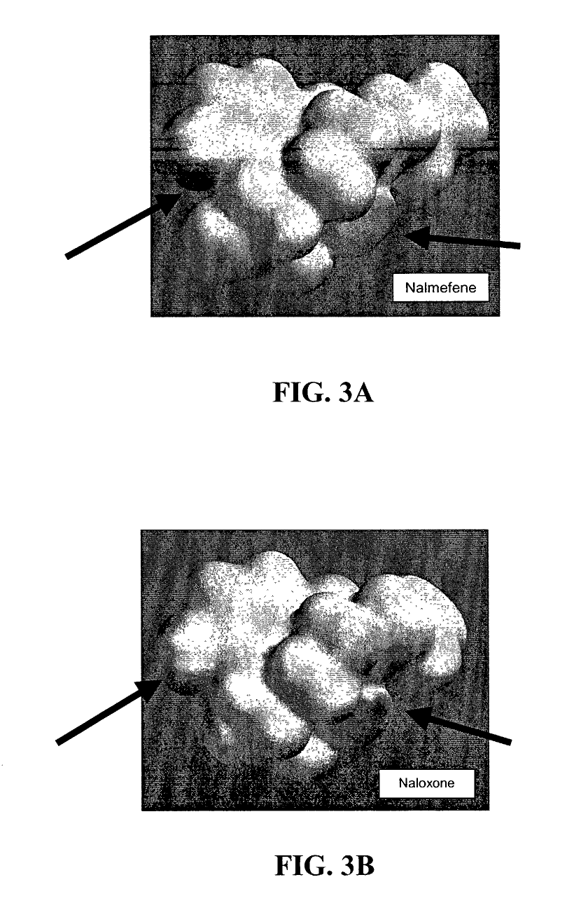

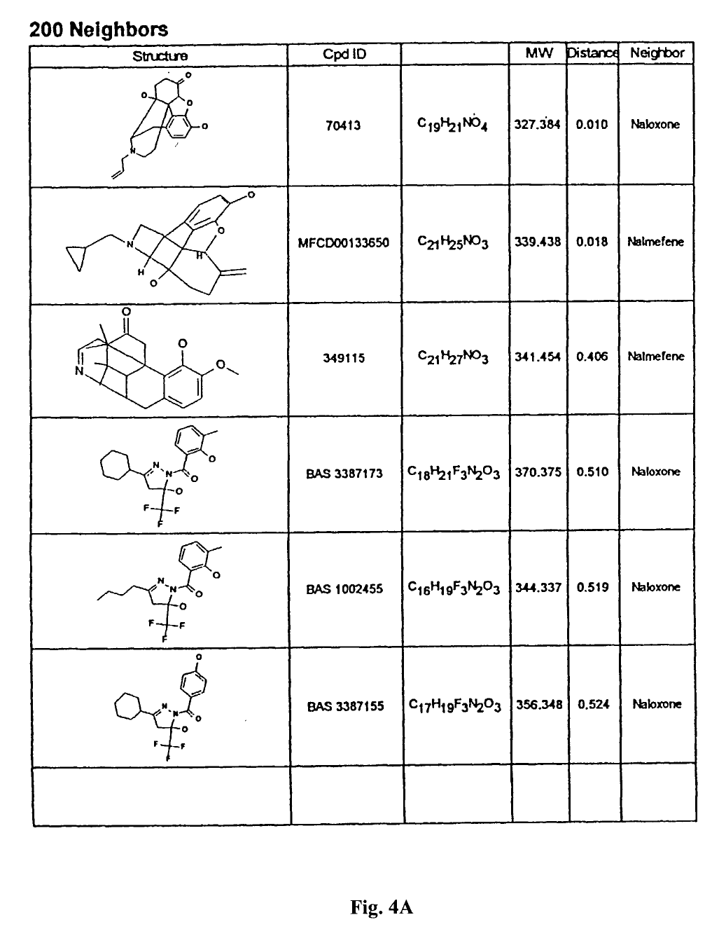

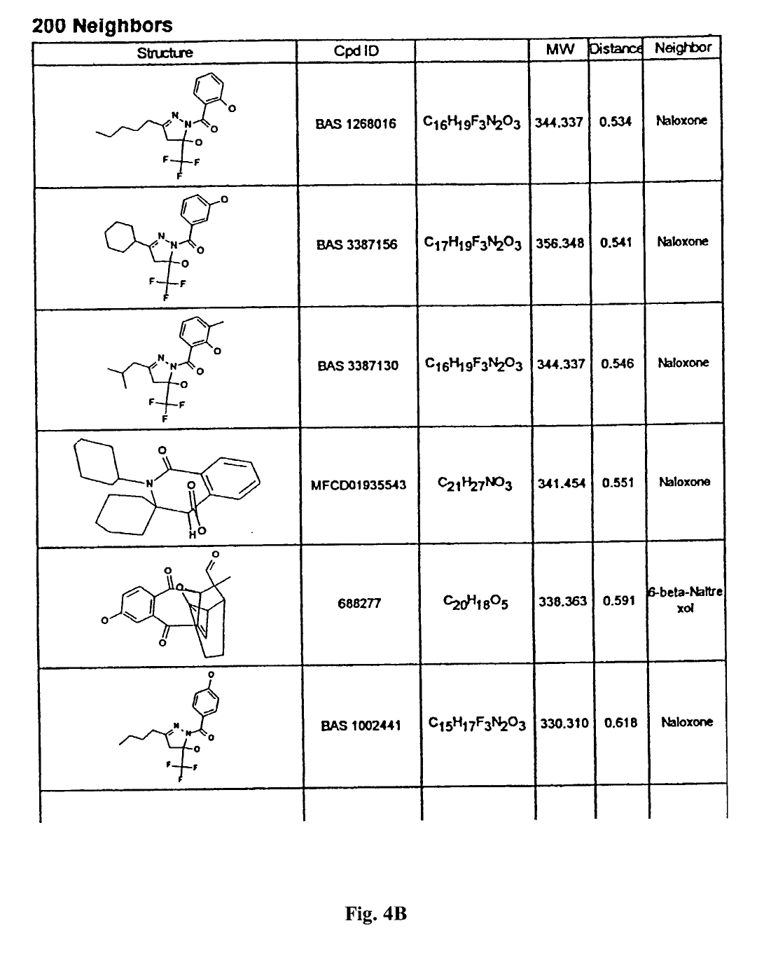

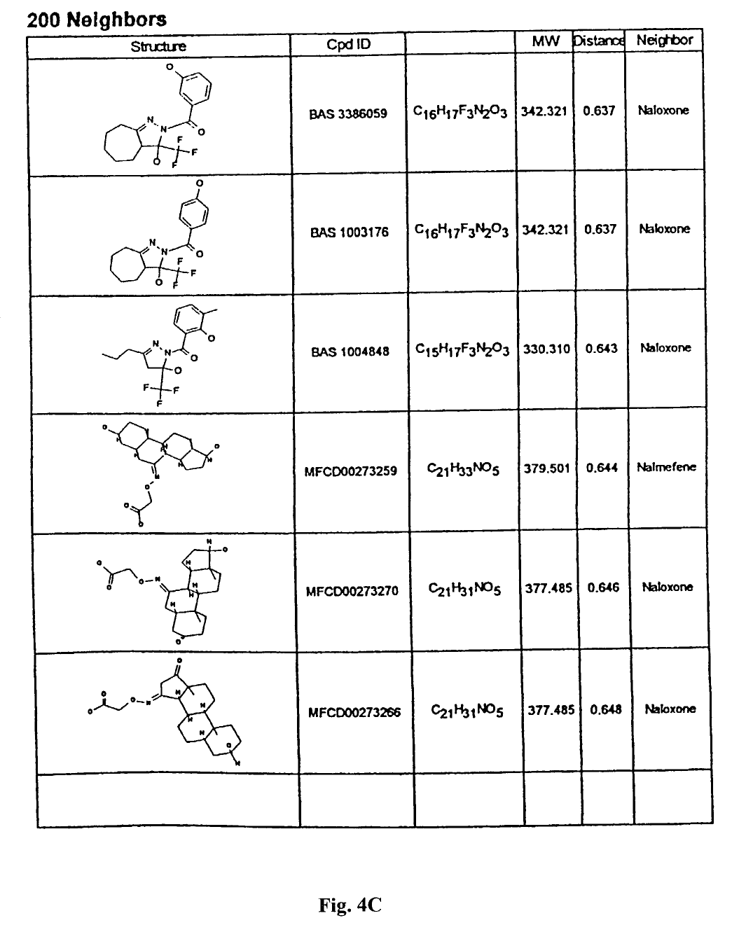

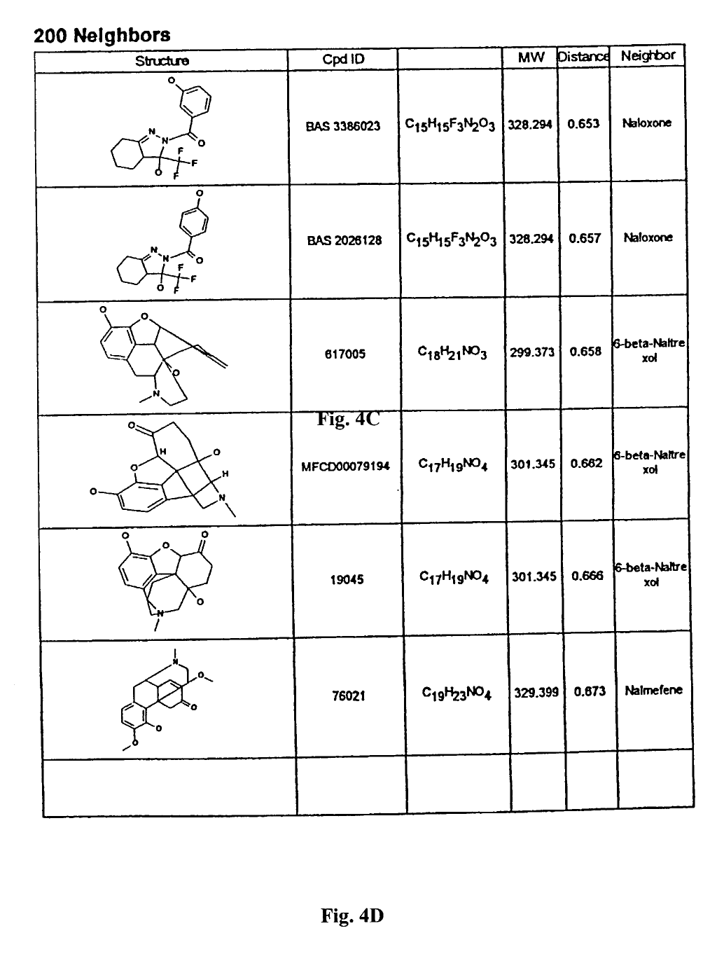

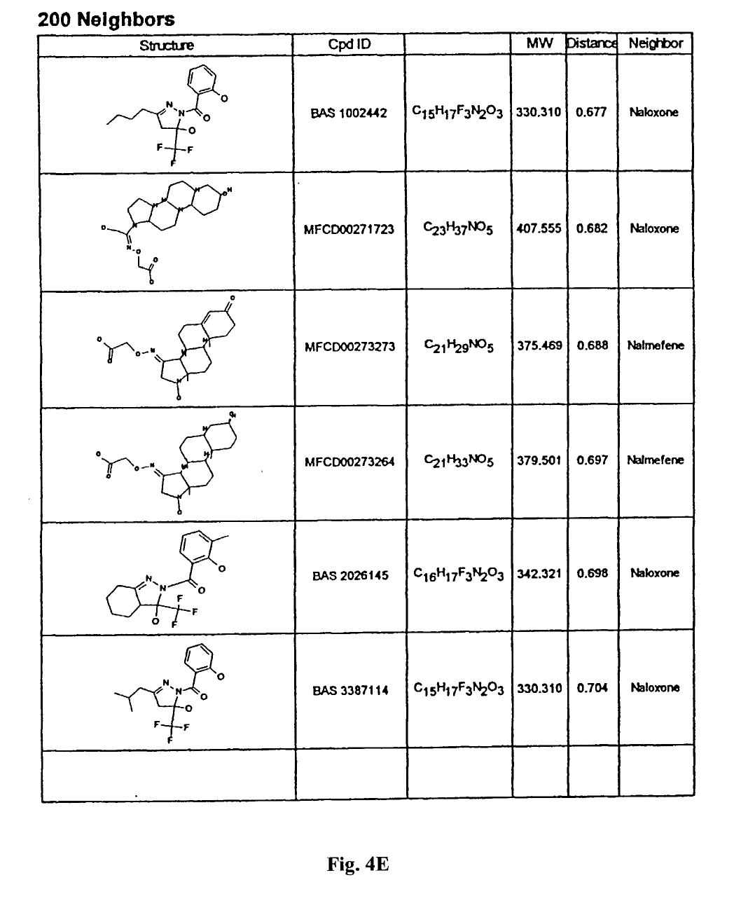

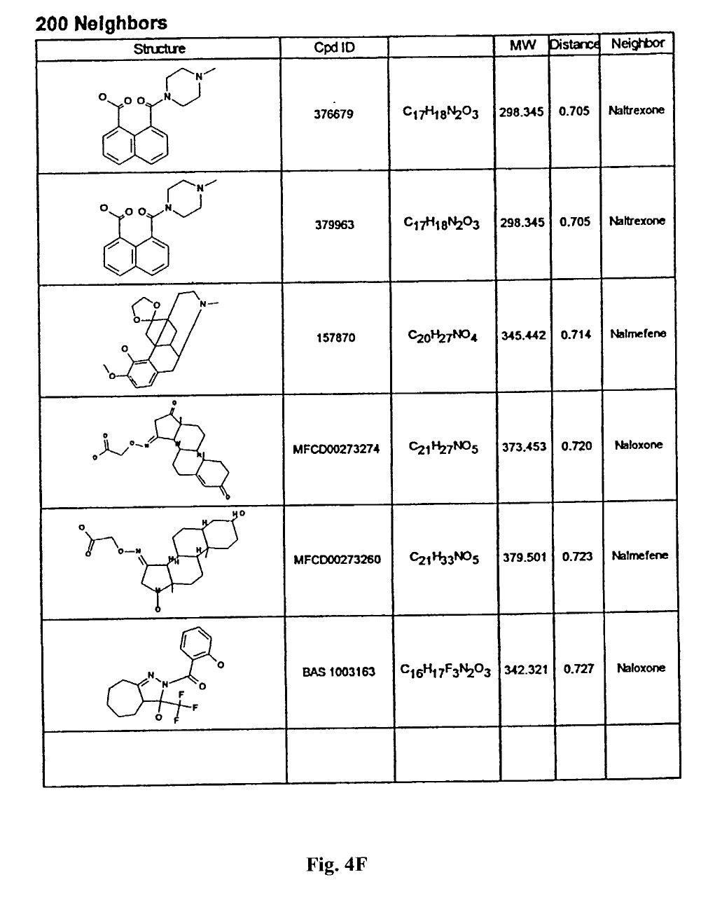

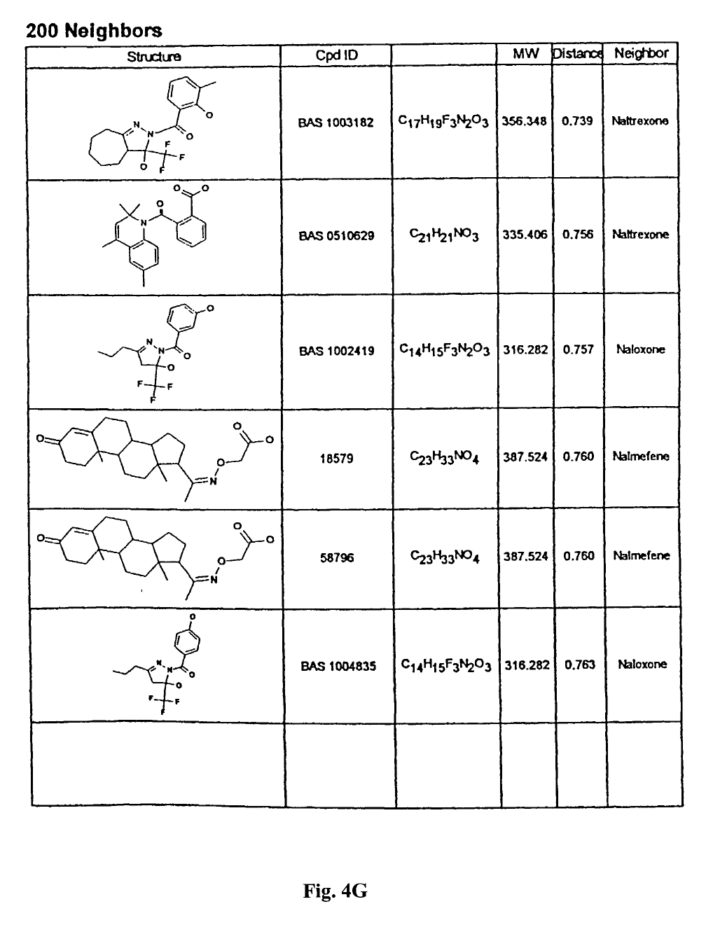

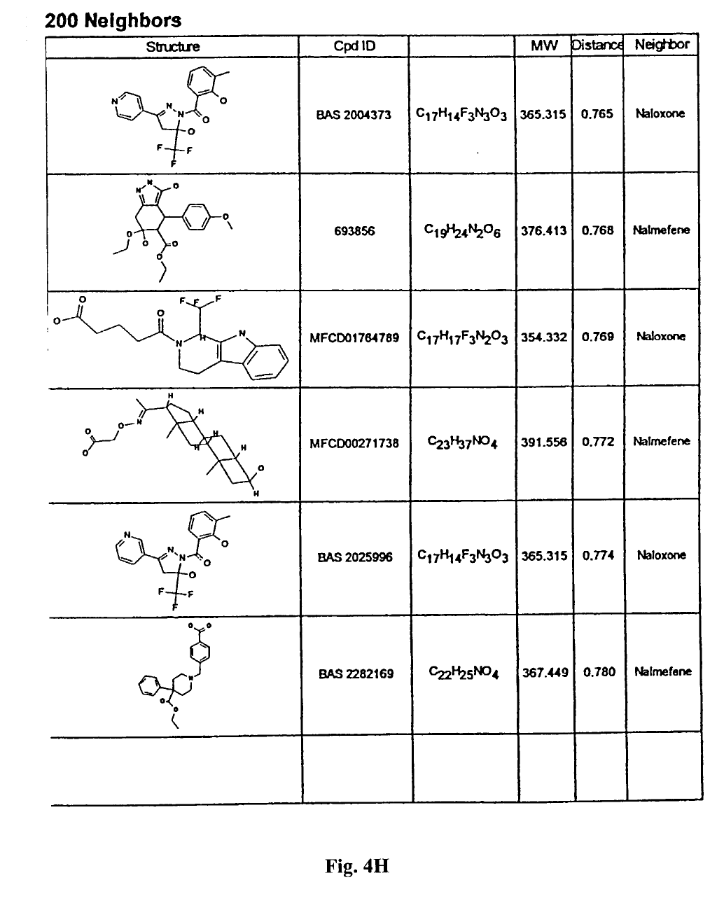

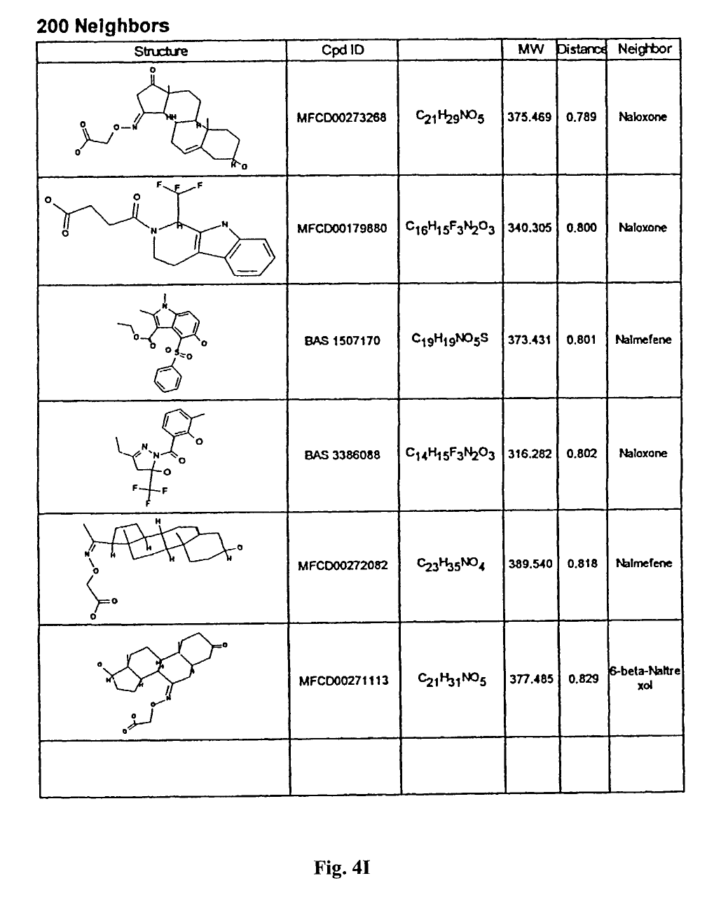

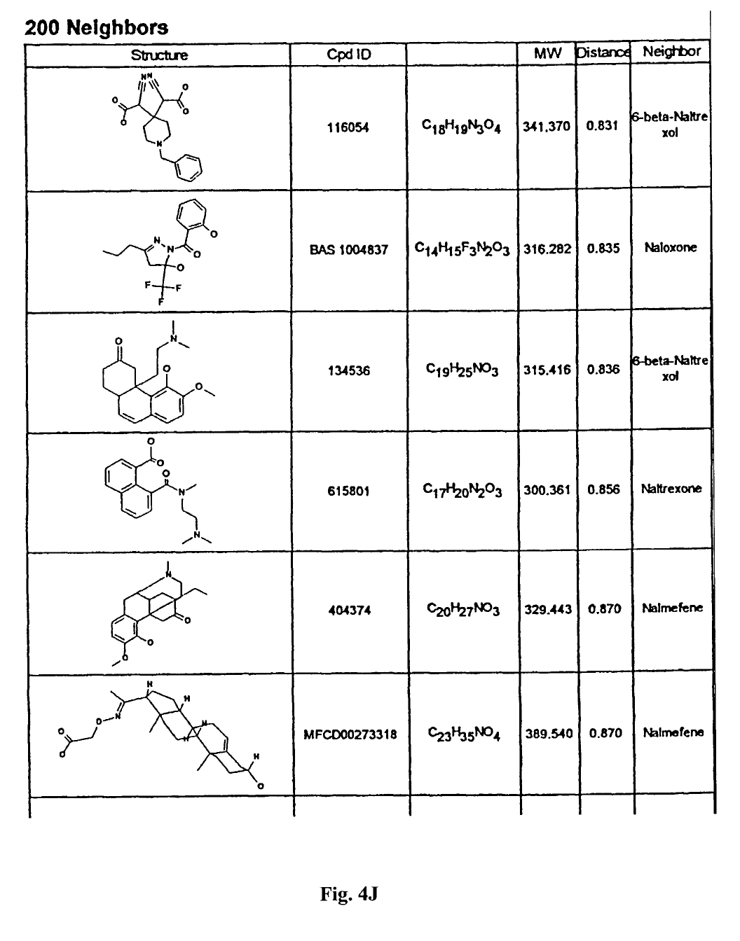

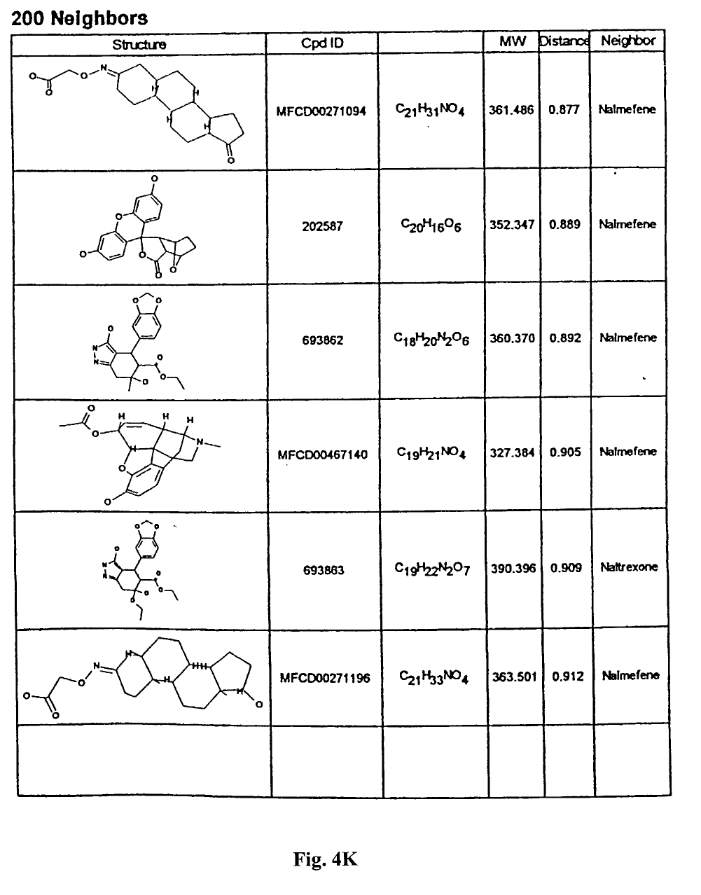

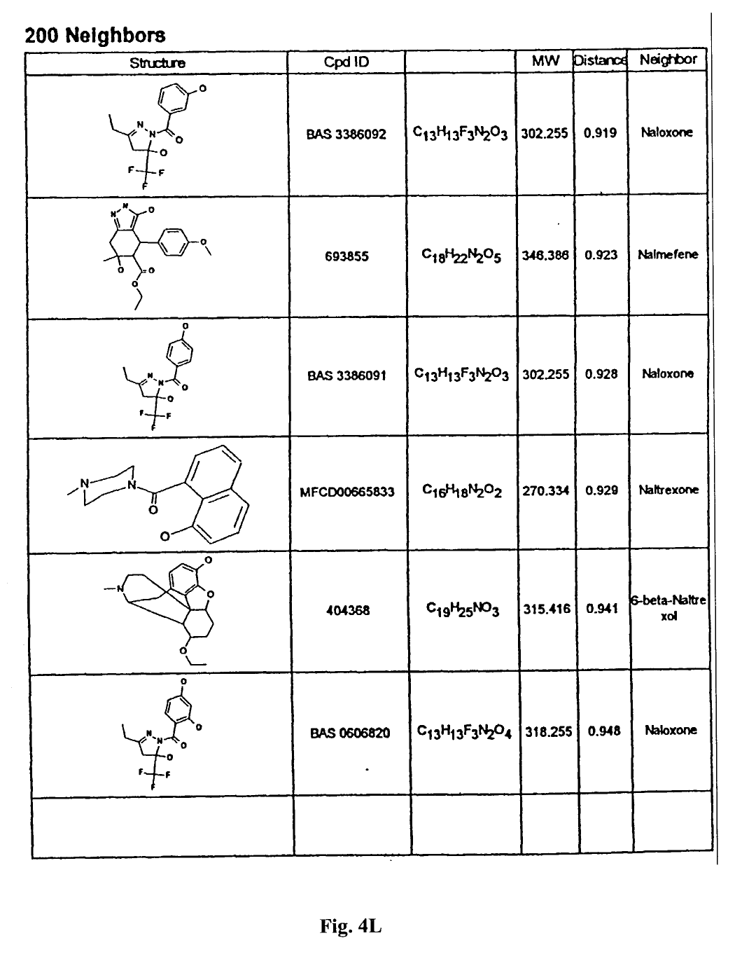

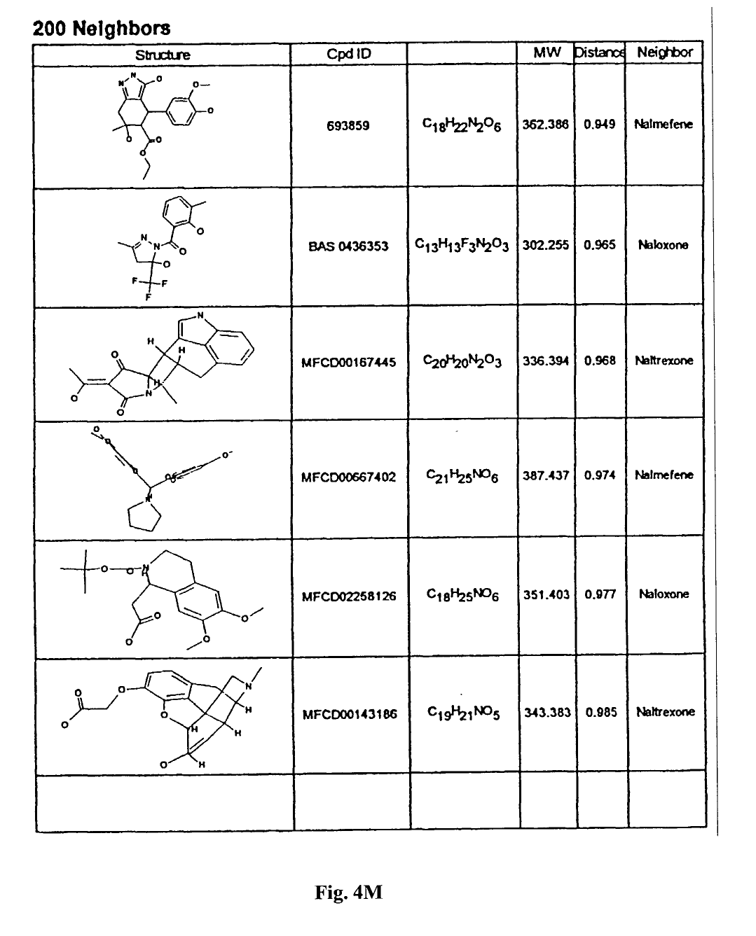

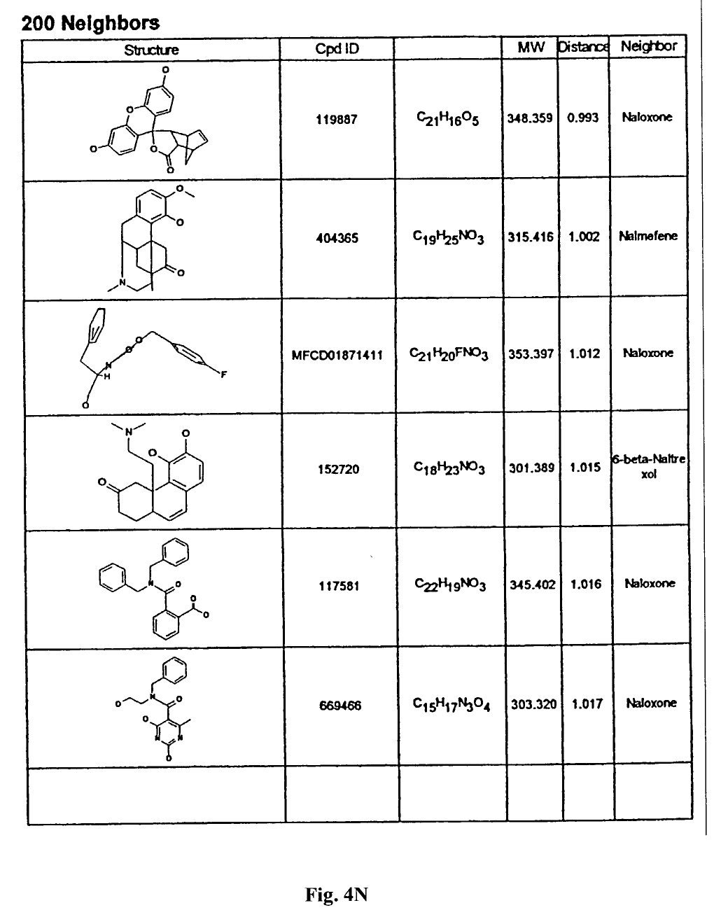

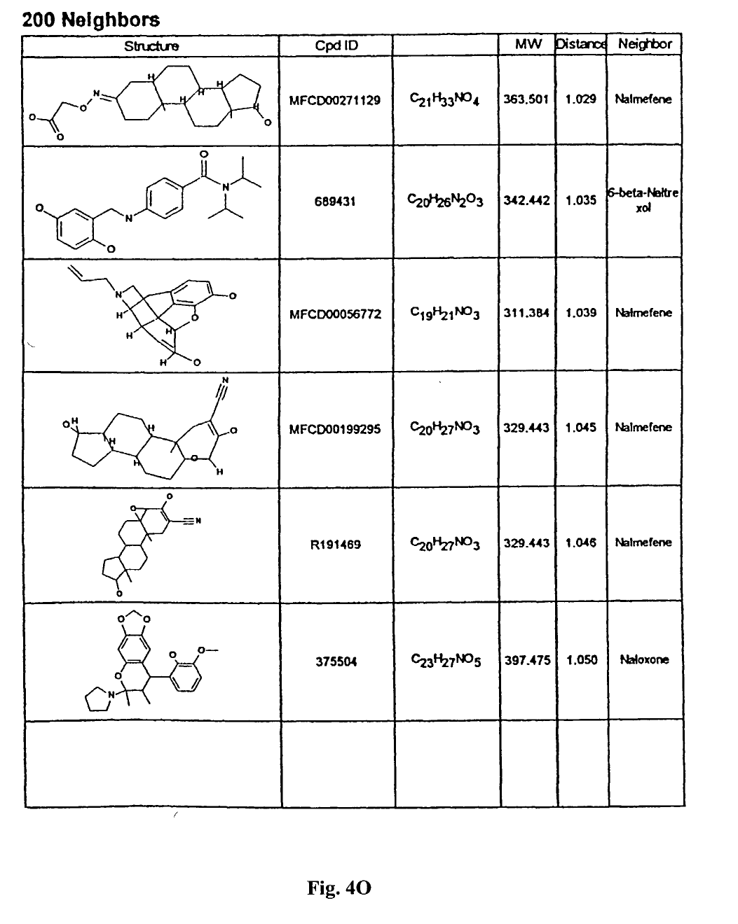

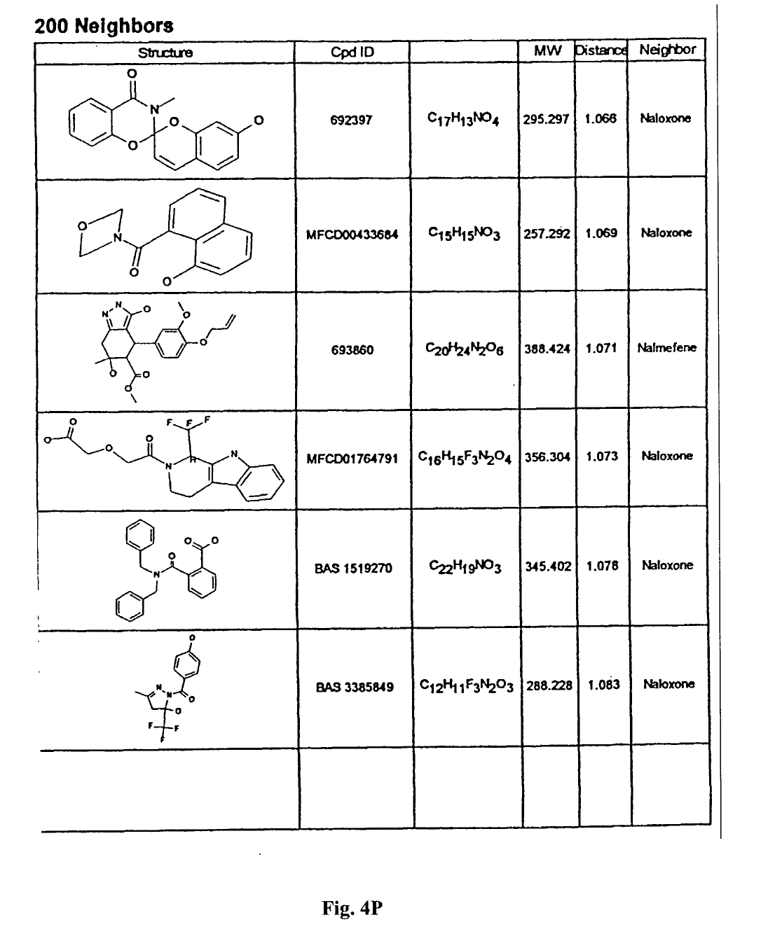

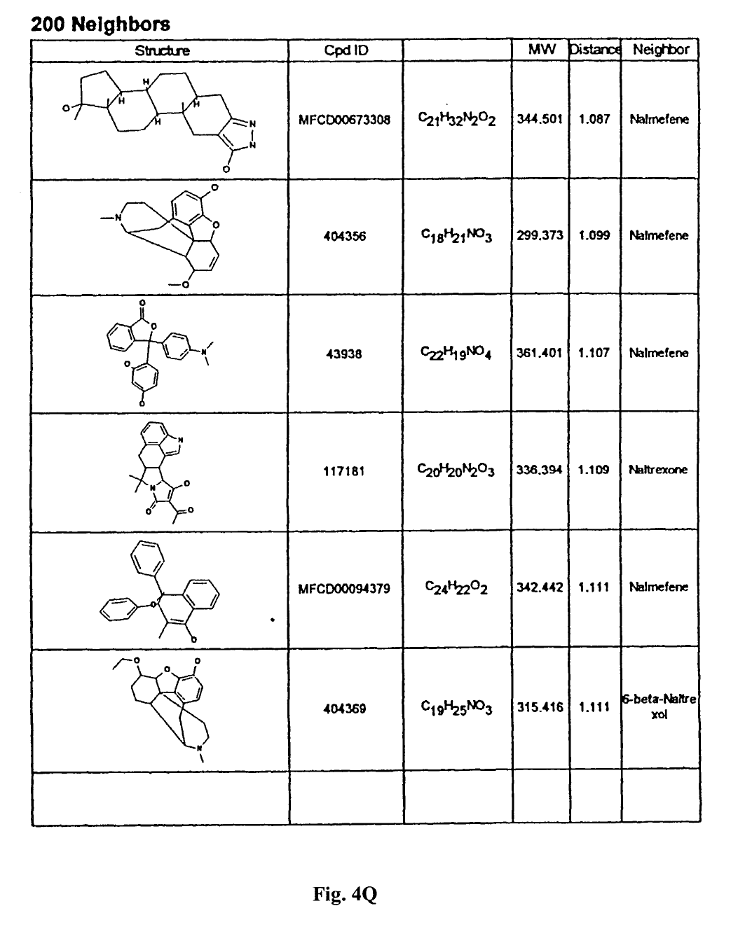

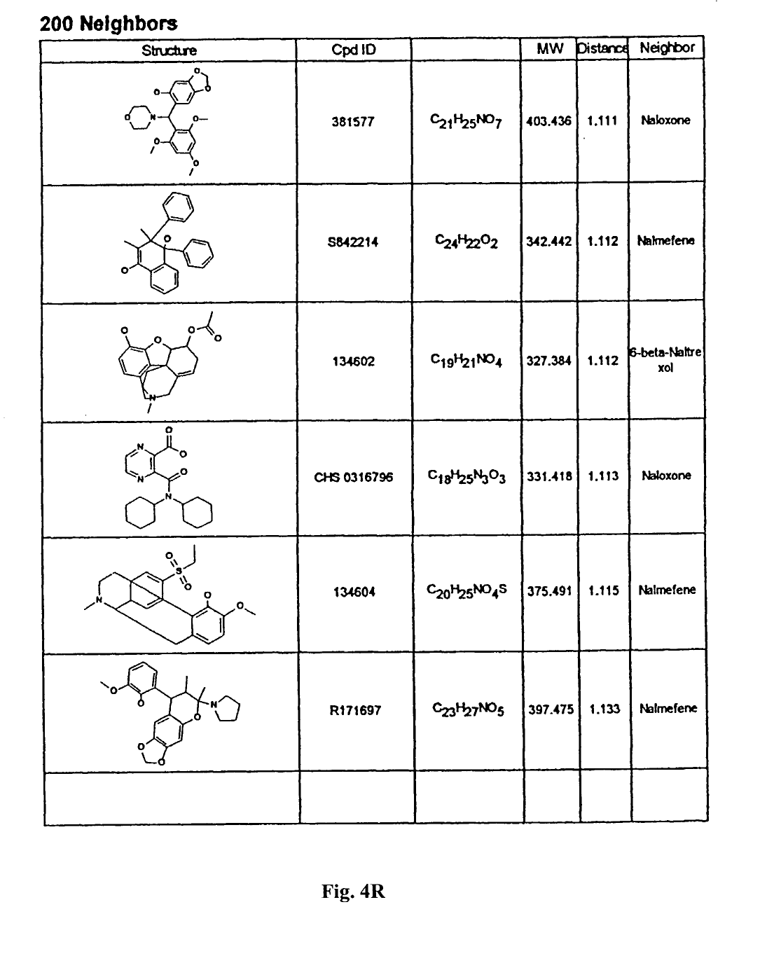

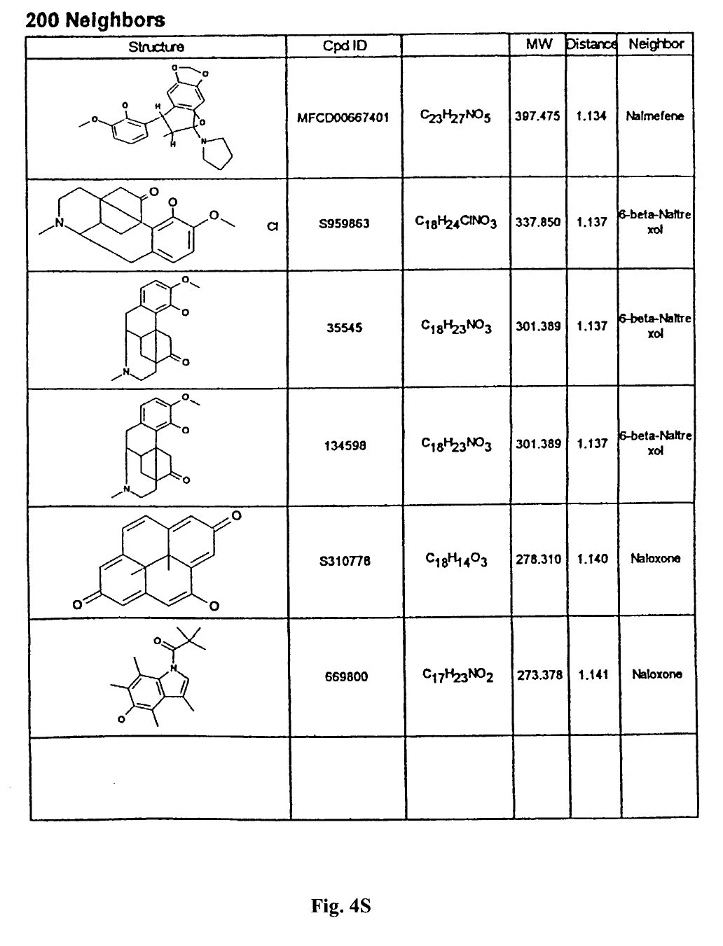

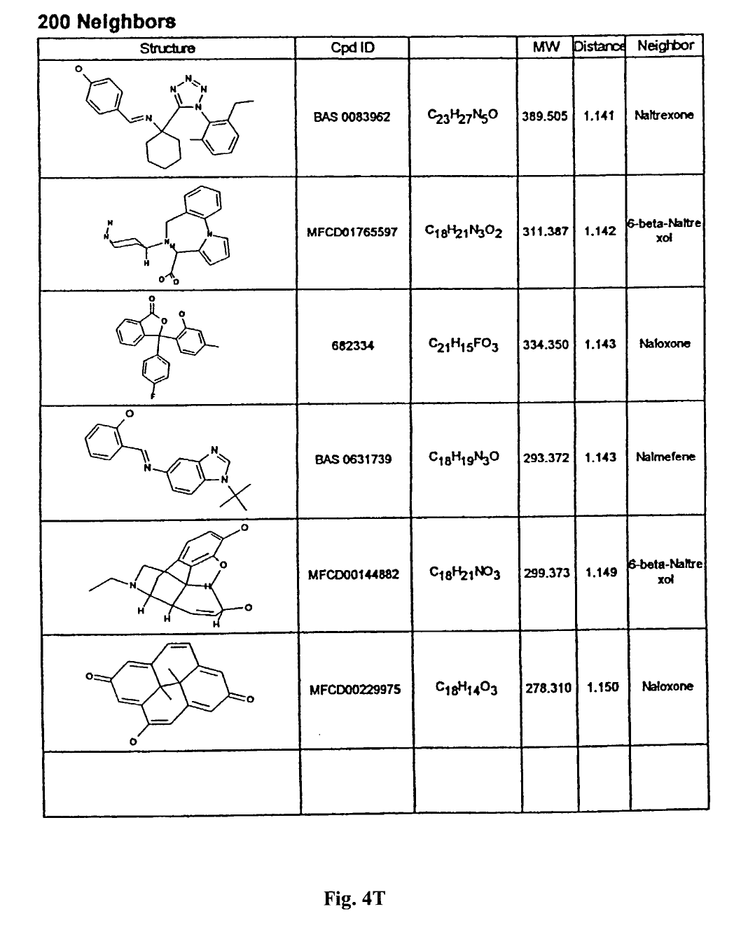

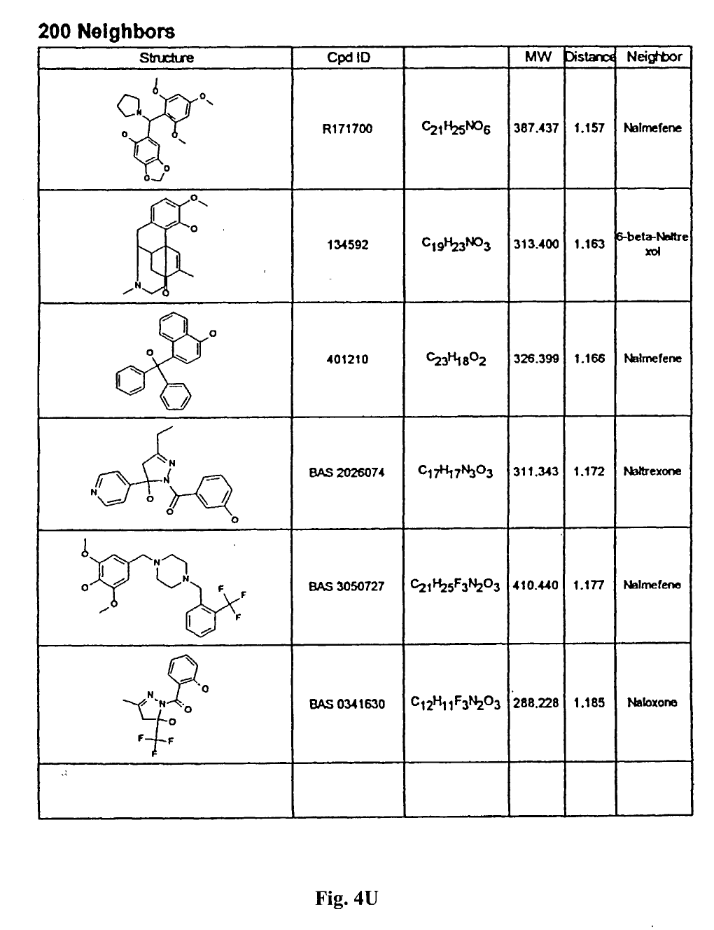

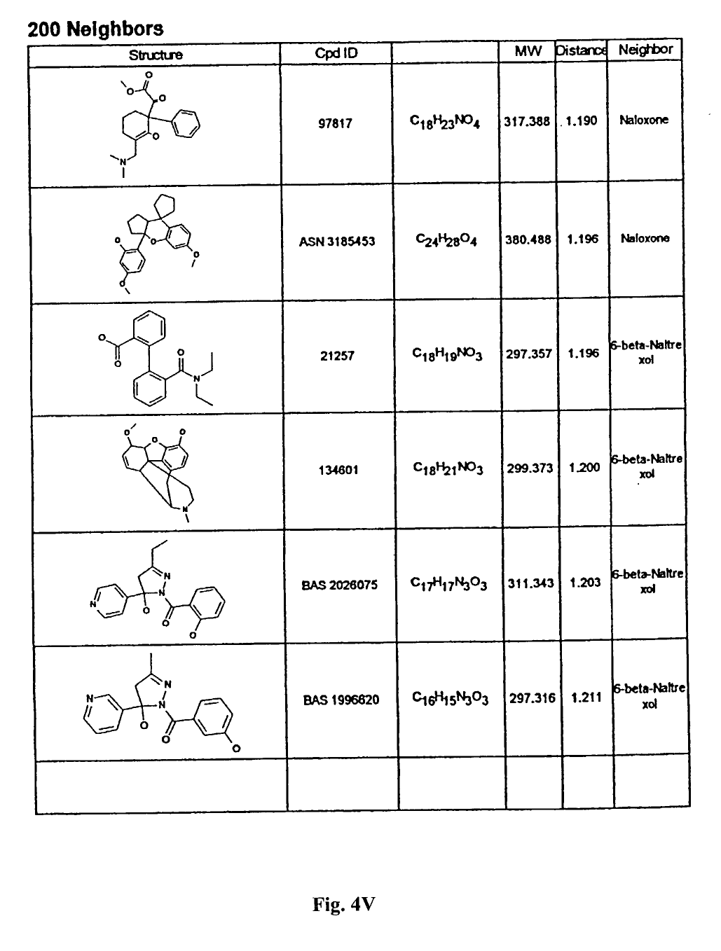

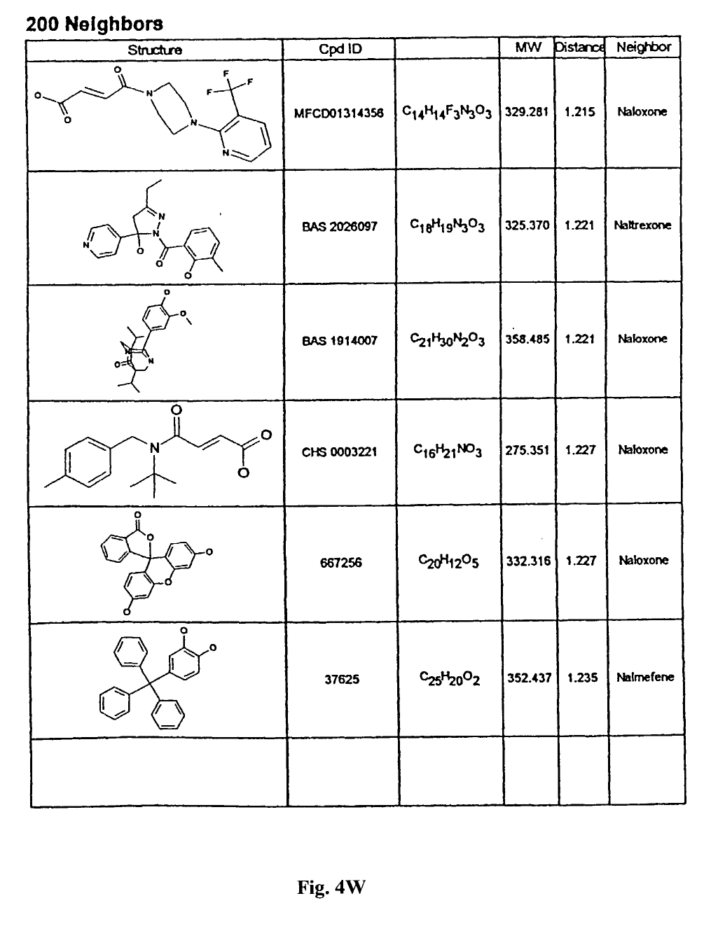

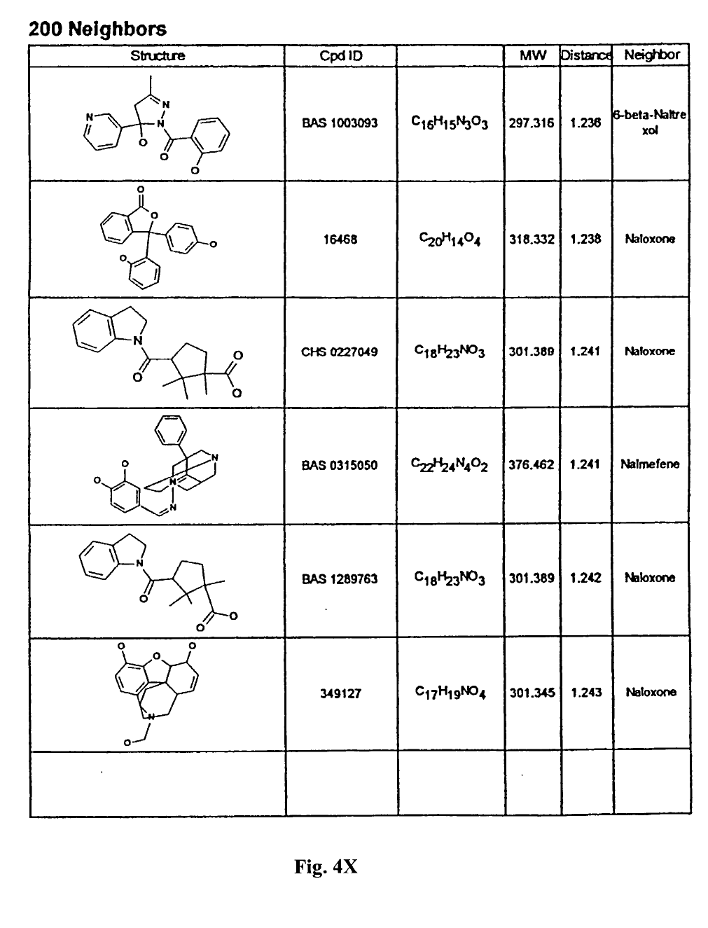

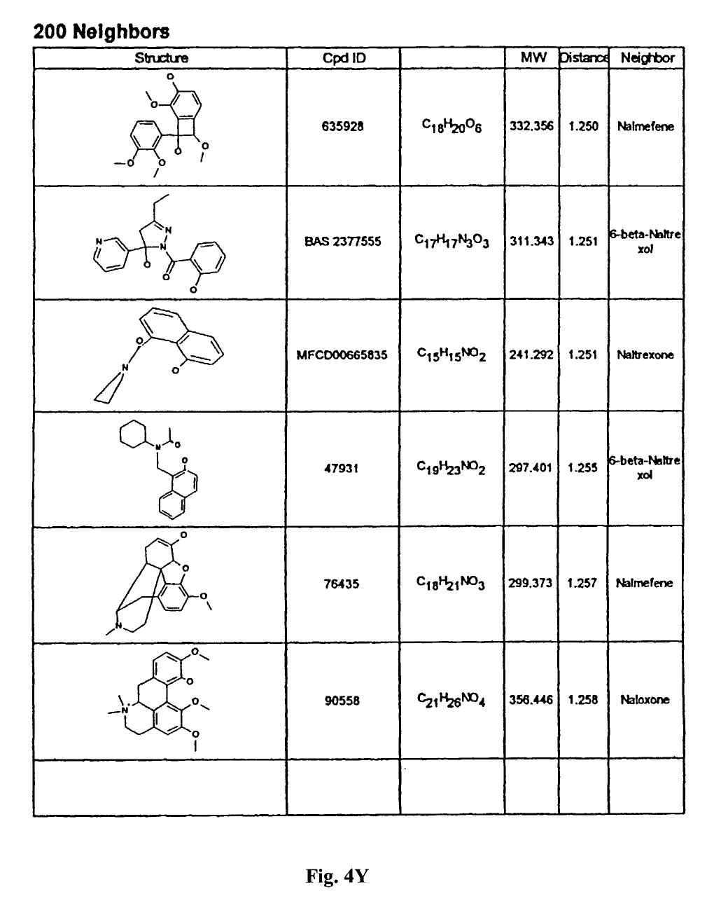

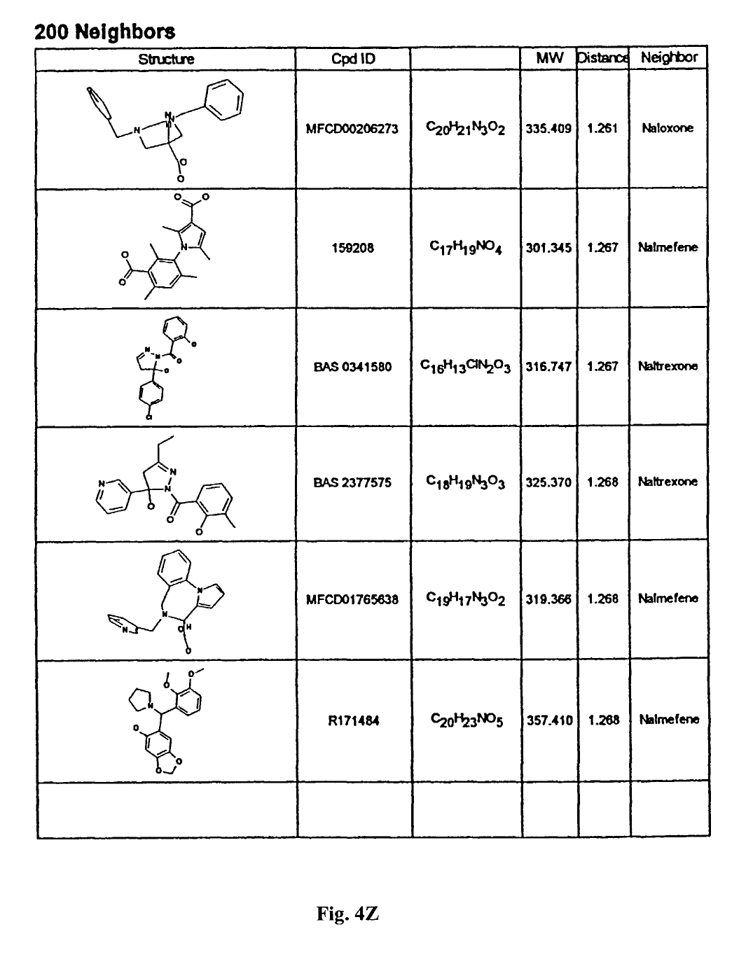

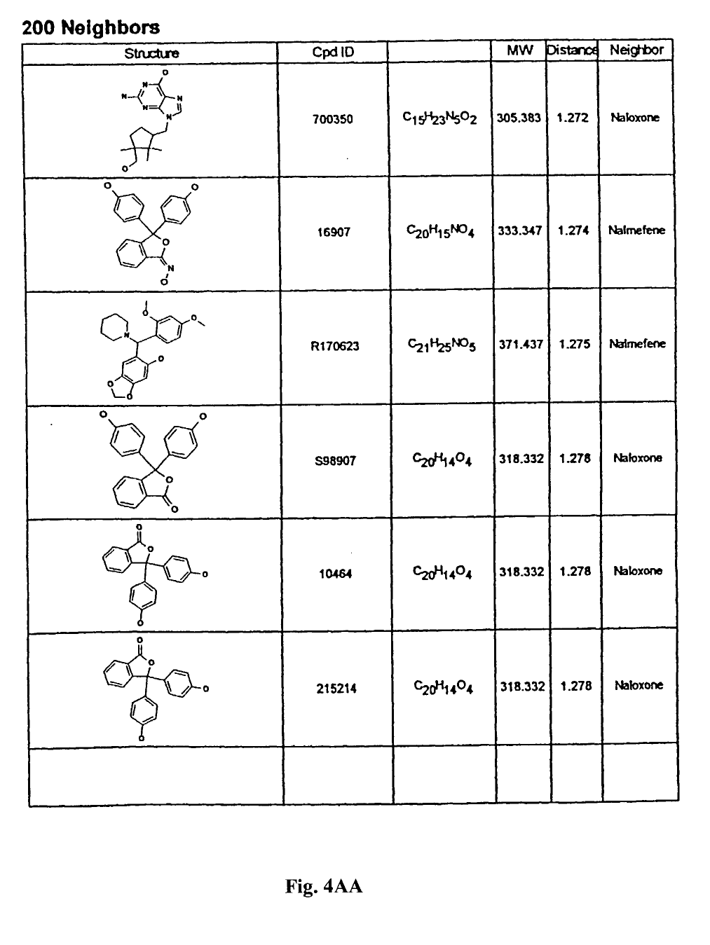

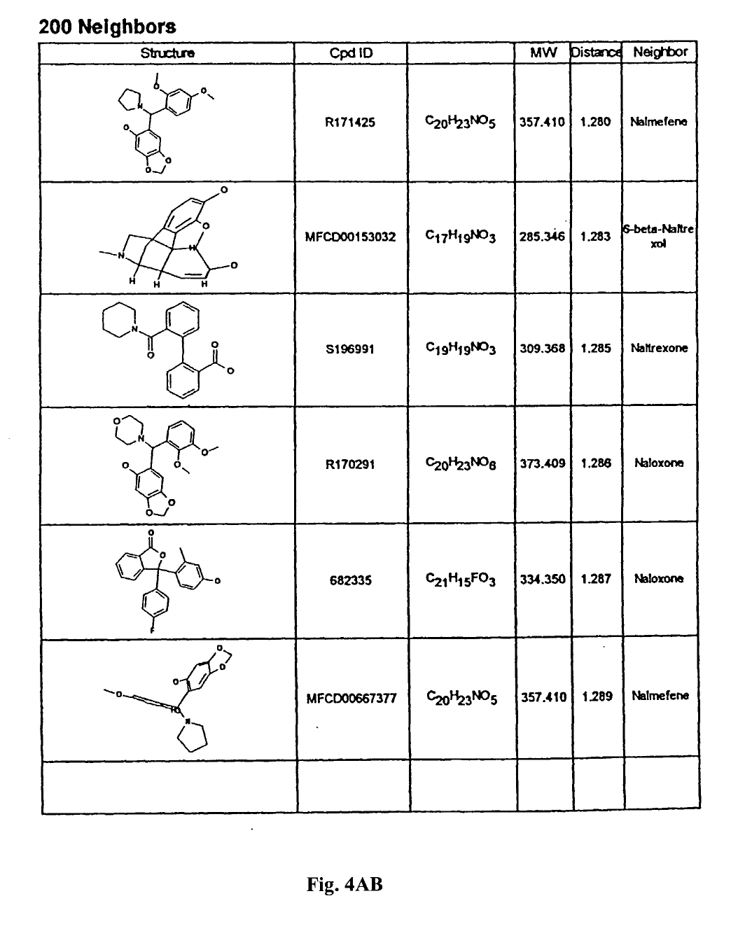

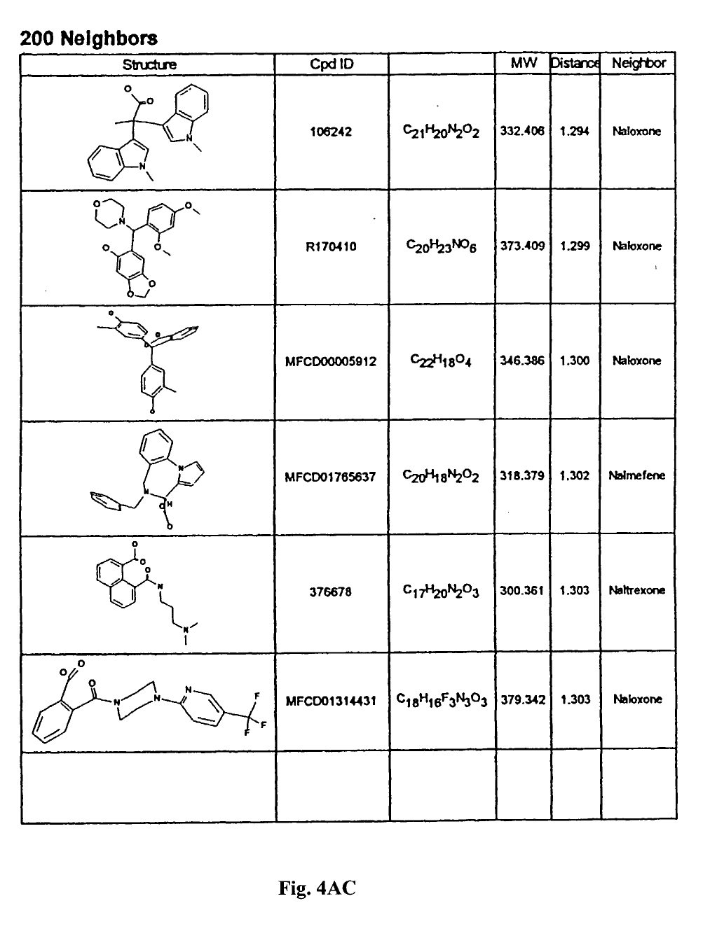

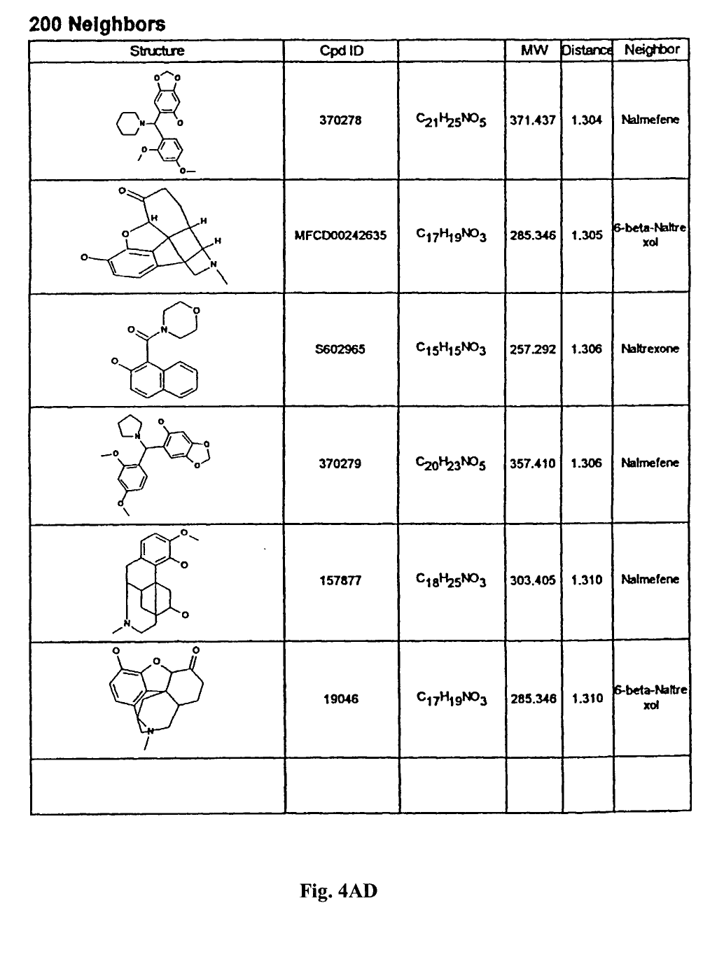

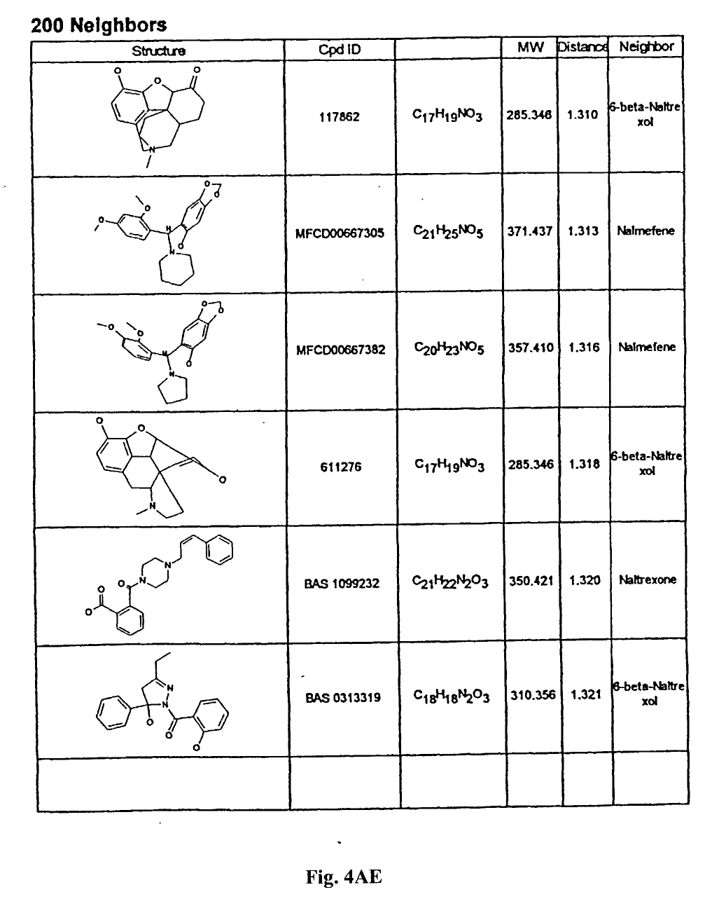

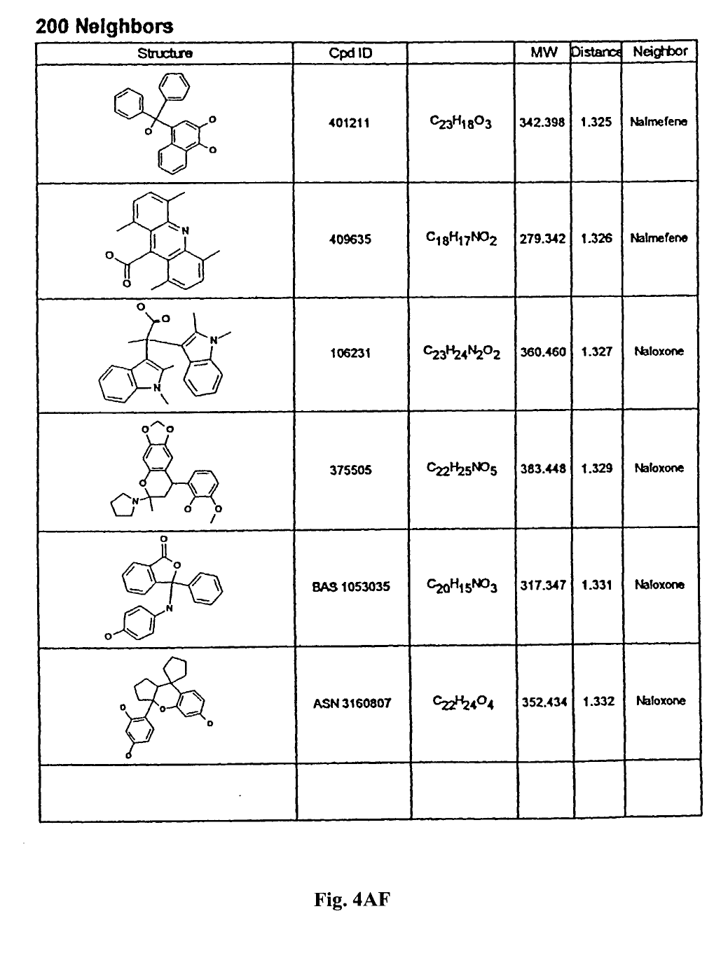

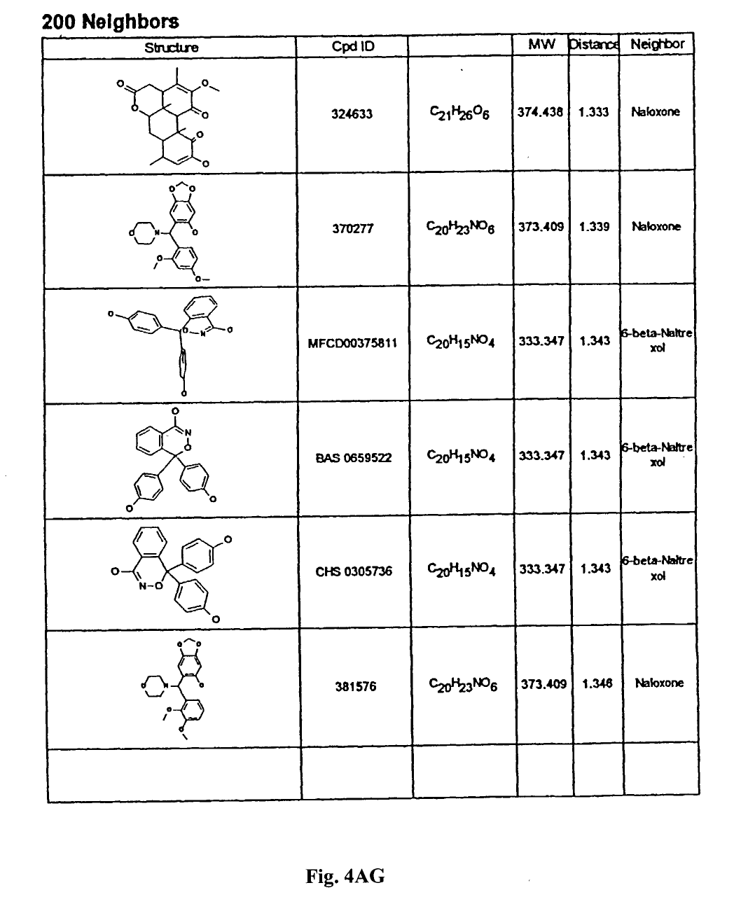

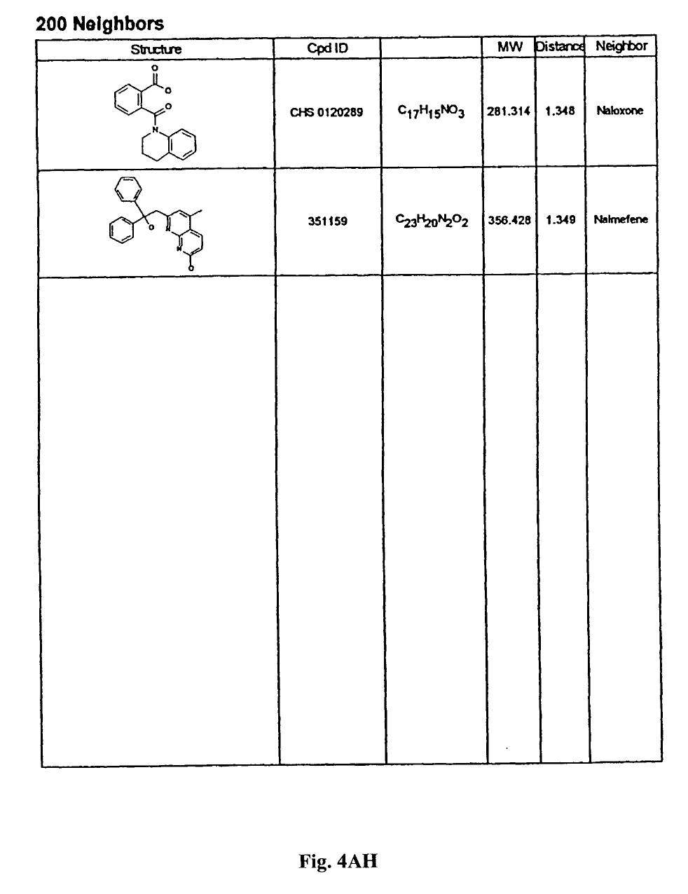

Molecular Orbital calculations were performed on the compounds using Spartan (Wavefunction, Inc.). There were no appreciable differences among the active compounds with respect to their electrostatic potentials. The electrostatic potential of nalmefene and naloxone are illustrated in FIGS. 3A and B respectively. The arrows indicate the hydroxyl group hydrogen-bond donor sites noted above.

Two views of an overlay of nalmefene and the low energy conformer of Kamm Compound 1 was prepared. The ring stacking structure predicted by Confort for the Kamm compounds embodies a conserved hydrophobic region shared by the both the Kamm compounds and the exemplary opioid compounds. The hydrogen-bond donor sites noted in the FIG. 3 overlap the predicted hydrogen bonding sites of the Kamm compound. The nalmefene furan ring oxygen overlays on an aromatic ring in Kamm Compound 1, suggesting that the oxygen atom is not necessary for this activity.

In silico analyses of chemical compounds were conducted as follows: Diversity estimations were made on nalmefene, naltrexone, naloxone, 6-β-naltrexol, and the 16 Kamm et al. structures using DiverseSolutions software from Tripos (R. S. Pearlman, UT-Austin). A chemistry space defined by approximately 900,000 chemical entities (several commercially available databases of compounds) was used as a reference. The commercial databases used as sources of the 900,000 chemical entities were MDL Information Systems (http://www.mdli.com), ACD Database (http://www.mdli.com/cgi/dynamic/product.html ?uid=$uid&key=$key&id=17), NCI (http://dtp.nci.nih.gov/docs/3d_database/structural_information/smiles_strings.html), Aldrich (http://www.sigma-aldrich.com/saws.nsf/ home?openframeset), ASINEx Ltd. (http://www.asinex.com), and Chemstar (http://www.chemstar.ru). A transporter-relevent subspace was determined based on the former chemistry space, using the “B→A/A→B” efflux ratios to represent the activities. In order to have sufficient data, the Kamm et al. data was combined with the high affinity/low capacity data provided for the exemplary opioid compounds. The 200 “nearest neighbors” are listed in Table 17 below. Note that in the Receptor-Relevant Subspace, the active compounds are focused in a small region of the overall chemistry space. Such compounds may be useful according to methods of the invention.

TABLE 17
200 Nearest Neighbors

| | Database | | | |
|---|---|---|---|---|
| Rank | I.D. # | Distance to Exemplary Compound | | |
| 1 | 70413 | 0.0096 | to | Naloxone |
| 2 | MFCD00133650 | 0.0184 | to | Nalmefene |
| 3 | 349115 | 0.4061 | to | Nalmefene |
| 4 | BAS 3387173 | 0.5101 | to | Naloxone |
| 5 | BAS 1002455 | 0.5195 | to | Naloxone |
| 6 | BAS 3387155 | 0.5243 | to | Naloxone |
| 7 | BAS 1268016 | 0.5345 | to | Naloxone |
| 8 | BAS 3387156 | 0.5412 | to | Naloxone |
| 9 | BAS 3387130 | 0.5462 | to | Naloxone |
| 10 | MFCD01935543 | 0.5507 | to | Naloxone |
| 11 | 688277 | 0.5913 | to | 6-β-Naltrexol |
| 12 | BAS 1002441 | 0.6179 | to | Naloxone |
| 13 | BAS 3386059 | 0.6369 | to | Naloxone |
| 14 | BAS 1003176 | 0.6370 | to | Naloxone |
| 15 | BAS 1004848 | 0.6434 | to | Naloxone |
| 16 | MFCD00273259 | 0.6436 | to | Nalmefene |
| 17 | MFCD00273270 | 0.6458 | to | Naloxone |
| 18 | MFCD00273266 | 0.6482 | to | Naloxone |
| 19 | BAS 3386023 | 0.6526 | to | Naloxone |
| 20 | BAS 2026128 | 0.6569 | to | Naloxone |
| 21 | 617005 | 0.6581 | to | 6-β-Naltrexol |
| 22 | MFCD00079194 | 0.6622 | to | 6-β-Naltrexol |
| 23 | 19045 | 0.6665 | to | 6-β-Naltrexol |
| 24 | 76021 | 0.6733 | to | Nalmefene |
| 25 | BAS 1002442 | 0.6770 | to | Naloxone |
| 26 | MFCD00271723 | 0.6822 | to | Naloxone |
| 27 | MFCD00273273 | 0.6884 | to | Nalmefene |
| 28 | MFCD00273264 | 0.6968 | to | Nalmefene |
| 29 | BAS 2026145 | 0.6977 | to | Naloxone |
| 30 | BAS 3387114 | 0.7036 | to | Naloxone |
| 31 | 376679 | 0.7051 | to | Naltrexone |
| 32 | 379963 | 0.7051 | to | Naltrexone |
| 33 | 157870 | 0.7144 | to | Nalmefene |
| 34 | MFCD00273274 | 0.7198 | to | Naloxone |
| 35 | MFCD00273260 | 0.7228 | to | Nalmefene |
| 36 | BAS 1003163 | 0.7272 | to | Naloxone |
| 37 | BAS 1003182 | 0.7388 | to | Naltrexone |
| 38 | BAS 0510629 | 0.7564 | to | Naltrexone |
| 39 | BAS 1002419 | 0.7571 | to | Naloxone |
| 40 | 18579 | 0.7600 | to | Nalmefene |
| 41 | 58796 | 0.7600 | to | Nalmefene |
| 42 | BAS 1004835 | 0.7634 | to | Naloxone |
| 43 | BAS 2004373 | 0.7646 | to | Naloxone |
| 44 | 693856 | 0.7680 | to | Nalmefene |
| 45 | MFCD01764789 | 0.7687 | to | Naloxone |
| 46 | MFCD00271738 | 0.7719 | to | Nalmefene |
| 47 | BAS 2025996 | 0.7741 | to | Naloxone |
| 48 | BAS 2282169 | 0.7798 | to | Nalmefene |
| 49 | MFCD00273268 | 0.7895 | to | Naloxone |
| 50 | MFCD00179880 | 0.7997 | to | Naloxone |
| 51 | BAS 1507170 | 0.8014 | to | Nalmefene |
| 52 | BAS 3386088 | 0.8017 | to | Naloxone |
| 53 | MFCD00272082 | 0.8183 | to | Nalmefene |
| 54 | MFCD00271113 | 0.8289 | to | 6-β-Naltrexol |
| 55 | 116054 | 0.8308 | to | 6-β-Naltrexol |
| 56 | BAS 1004837 | 0.8352 | to | Naloxone |
| 57 | 134536 | 0.8364 | to | 6-β-Naltrexol |
| 58 | 615801 | 0.8556 | to | Naltrexone |
| 59 | 404374 | 0.8695 | to | Nalmefene |
| 60 | MFCD00273318 | 0.8697 | to | Nalmefene |
| 61 | MFCD00271094 | 0.8774 | to | Nalmefene |
| 62 | 202587 | 0.8895 | to | Nalmefene |
| 63 | 693862 | 0.8919 | to | Nalmefene |
| 64 | MFCD00467140 | 0.9049 | to | Nalmefene |
| 65 | 693863 | 0.9093 | to | Naltrexone |
| 66 | MFCD00271196 | 0.9123 | to | Nalmefene |
| 67 | BAS 3386092 | 0.9195 | to | Naloxone |
| 68 | 693855 | 0.9235 | to | Nalmefene |
| 69 | BAS 3386091 | 0.9278 | to | Naloxone |
| 70 | MFCD00665833 | 0.9291 | to | Naltrexone |
| 71 | 404368 | 0.9412 | to | 6-β-Naltrexol |
| 72 | BAS 0606820 | 0.9478 | to | Naloxone |
| 73 | 693859 | 0.9485 | to | Nalmefene |
| 74 | BAS 0436353 | 0.9653 | to | Naloxone |
| 75 | MFCD00167445 | 0.9681 | to | Naltrexone |
| 76 | MFCD00667402 | 0.9742 | to | Nalmefene |
| 77 | MFCD002258126 | 0.9767 | to | Naloxone |
| 78 | MFCD00143186 | 0.9850 | to | Naltrexone |
| 79 | 119887 | 0.9932 | to | Naloxone |
| 80 | 404365 | 1.0016 | to | Nalmefene |
| 81 | MFCD01871411 | 1.0116 | to | Naloxone |
| 82 | 152720 | 1.0147 | to | 6-β-Naltrexol |
| 83 | 117581 | 1.0164 | to | Naloxone |
| 84 | 669466 | 1.0171 | to | Naloxone |
| 85 | MFCD00271129 | 1.0287 | to | Nalmefene |
| 86 | 689431 | 1.0350 | to | 6-β-Naltrexol |
| 87 | MFCD00056772 | 1.0390 | to | Nalmefene |
| 88 | MFCD00199295 | 1.0449 | to | Nalmefene |
| 89 | R191469 | 1.0457 | to | Nalmefene |
| 90 | 375504 | 1.0503 | to | Naloxone |
| 91 | 692397 | 1.0656 | to | Naloxone |
| 92 | MFCD00433684 | 1.0691 | to | Naloxone |
| 93 | 693860 | 1.0709 | to | Nalmefene |
| 94 | MFCD01764791 | 1.0725 | to | Naloxone |
| 95 | BAS 1519270 | 1.0776 | to | Naloxone |
| 96 | BAS 3385849 | 1.0828 | to | Naloxone |
| 97 | MFCD00673308 | 1.0866 | to | Nalmefene |
| 98 | 404356 | 1.0990 | to | Nalmefene |
| 99 | 43938 | 1.1067 | to | Nalmefene |
| 100 | 117181 | 1.1092 | to | Naltrexone |
| 101 | MFCD00094379 | 1.1109 | to | Nalmefene |
| 102 | 404369 | 1.1109 | to | 6-β-Naltrexol |
| 103 | 381577 | 1.1111 | to | Naloxone |
| 104 | S842214 | 1.1117 | to | Nalmefene |
| 105 | 134602 | 1.1123 | to | 6-β-Naltrexol |
| 108 | CHS 0316796 | 1.1130 | to | Naloxone |
| 107 | 134604 | 1.1147 | to | Nalmefene |
| 108 | R171697 | 1.1334 | to | Nalmefene |
| 109 | MFCD00667401 | 1.1343 | to | Nalmefene |
| 110 | S959863 | 1.1367 | to | 6-β-Naltrexol |
| 111 | 35545 | 1.1369 | to | 6-β-Naltrexol |
| 112 | 134598 | 1.1369 | to | 6-β-Naltrexol |
| 113 | S310778 | 1.1403 | to | Naloxone |
| 114 | 669800 | 1.1408 | to | Naloxone |
| 115 | BAS 0083962 | 1.1413 | to | Naltrexone |
| 116 | MFCD01765597 | 1.1424 | to | 6-β-Naltrexol |
| 117 | 682334 | 1.1427 | to | Naloxone |
| 118 | BAS 0631739 | 1.1428 | to | Nalmefene |
| 119 | MFCD00144882 | 1.1486 | to | 6-β-Naltrexol |
| 120 | MFCD00229975 | 1.1497 | to | Naloxone |
| 121 | R171700 | 1.1568 | to | Nalmefene |
| 122 | 134592 | 1.1633 | to | 6-β-Naltrexol |
| 123 | 401210 | 1.1662 | to | Nalmefene |
| 124 | BAS 2026074 | 1.1715 | to | Naltrexone |
| 125 | BAS 3050727 | 1.1767 | to | Nalmefene |
| 126 | BAS 0341630 | 1.1851 | to | Naloxone |
| 127 | 97817 | 1.1901 | to | Naloxone |
| 128 | ASN 3185453 | 1.1958 | to | Naloxone |
| 129 | 21257 | 1.1962 | to | 6-β-Naltrexol |
| 130 | 134601 | 1.2005 | to | 6-β-Naltrexol |
| 131 | BAS 2026075 | 1.2027 | to | 6-β-Naltrexol |
| 132 | BAS 1996620 | 1.2114 | to | 6-β-Naltrexol |
| 133 | MFCD01314356 | 1.2147 | to | Naloxone |
| 134 | BAS 2026097 | 1.2207 | to | Naltrexone |
| 135 | BAS 1914007 | 1.2210 | to | Naloxone |
| 136 | CHS 0003221 | 1.2266 | to | Naloxone |
| 137 | 667258 | 1.2274 | to | Naloxone |
| 138 | 37625 | 1.2351 | to | Nalmefene |
| 139 | BAS 1003093 | 1.2362 | to | 6-β-Naltrexol |
| 140 | 16468 | 1.2380 | to | Naloxone |
| 141 | CHS 0227049 | 1.2409 | to | Naloxone |
| 142 | BAS 0315050 | 1.2410 | to | Nalmefene |
| 143 | BAS 1289763 | 1.2421 | to | Naloxone |
| 144 | 349127 | 1.2429 | to | Naloxone |
| 145 | 635928 | 1.2496 | to | Nalmefene |
| 146 | BAS 2377555 | 1.2507 | to | 6-β-Naltrexol |
| 147 | MFCD00665835 | 1.2508 | to | Naltrexone |
| 148 | 47931 | 1.2547 | to | 6-β-Naltrexol |
| 149 | 76435 | 1.2572 | to | Nalmefene |
| 150 | 90558 | 1.2581 | to | Naloxone |
| 151 | MFCD00206273 | 1.2608 | to | Naloxone |
| 152 | 159208 | 1.2670 | to | Nalmefene |
| 153 | BAS 0341580 | 1.2672 | to | Naltrexone |
| 154 | BAS 2377575 | 1.2678 | to | Naltrexone |
| 155 | MFCD01765638 | 1.2681 | to | Nalmefene |
| 156 | R171484 | 1.2684 | to | Nalmefene |
| 157 | 700350 | 1.2716 | to | Naloxone |
| 158 | 16907 | 1.2740 | to | Nalmefene |
| 159 | R170623 | 1.2754 | to | Nalmefene |
| 160 | S98907 | 1.2776 | to | Naloxone |
| 161 | 10464 | 1.2777 | to | Naloxone |
| 162 | 215214 | 1.2777 | to | Naloxone |
| 163 | R171425 | 1.2802 | to | Nalmefene |
| 164 | MFCD00153032 | 1.2831 | to | 6-β-Naltrexol |
| 165 | S196991 | 1.2850 | to | Naltrexone |
| 166 | R170291 | 1.2863 | to | Naloxone |
| 167 | 682335 | 1.2867 | to | Naloxone |
| 168 | UFCD00667377 | 1.2889 | to | Nalmefene |
| 169 | 106242 | 12944 | to | Naloxone |
| 170 | R170410 | 1.2989 | to | Naloxone |
| 171 | MFCD0005912 | 1.2996 | to | Naloxone |
| 172 | MFCD01765637 | 1.3018 | to | Nalmefene |
| 173 | 376678 | 1.3028 | to | Naltrexone |
| 174 | MFCD01314431 | 1.3031 | to | Naloxone |
| 175 | 370278 | 1.3040 | to | Nalmefene |
| 176 | MFCD00242635 | 1.3054 | to | 6-β-Naltrexol |
| 177 | S602965 | 1.3058 | to | Naltrexone |
| 178 | 370279 | 1.3063 | to | Nalmefene |
| 179 | 157877 | 1.3099 | to | Nalmefene |
| 180 | 19046 | 1.3103 | to | 6-β-Naltrexol |
| 181 | 117862 | 1.3103 | to | 6-β-Naltrexol |
| 182 | MFCD00667305 | 1.3134 | to | Nalmefene |
| 183 | MFCD00667382 | 1.3161 | to | Nalmefene |
| 184 | 611276 | 1.3178 | to | 6-β-Naltrexol |
| 185 | BAS 1099232 | 1.3197 | to | Naltrexone |
| 186 | BAS 0313319 | 1.3206 | to | 6-β-Naltrexol |
| 187 | 401211 | 1.3254 | to | Nalmefene |
| 188 | 409635 | 1.3263 | to | Nalmefene |
| 189 | 106231 | 1.3271 | to | Naloxone |
| 190 | 375505 | 1.3289 | to | Naloxone |
| 191 | BAS 1053035 | 1.3309 | to | Naloxone |
| 192 | ASN 3160807 | 1.3316 | to | Naloxone |
| 193 | 324633 | 1.3331 | to | Naloxone |
| 194 | 370277 | 1.3392 | to | Naloxone |
| 195 | MFCD00375811 | 1.3428 | to | 6-β-Naltrexol |
| 196 | CHS 0305736 | 1.3435 | to | 6-β-Naltrexol |
| 197 | BAS 0659522 | 1.3435 | to | 6-β-Naltrexol |
| 198 | 381576 | 1.3461 | to | Naloxone |
| 199 | CHS 0120289 | 1.3484 | to | Naloxone |
| 200 | 351159 | 1.3490 | to | Nalmefene |

The distance between the hydroxyl groups in the pharmacophore (“H” of OH to “H” of OH) is approximately 7.4 Å. The equivalent distance in “Kamm 1” is ˜7.7 Å. These stances are to the Hydrogen atoms, rather than the H-bond acceptors in the binding site. The N-substituent lengths of nalmefene (from N to terminal Carbons) are ˜3.9 Å and ˜3.5 Å. N-substituent length of naloxone (from N to terminal Carbon) is ˜3.4 Å.

Three-dimensional coordinates of naltrexone are provided in Table 18.

TABLE 18
Three-Dimensional Coordinates

| ATOM | X | Y | Z | Type | Charge |
|---|---|---|---|---|---|
| C1 | −0.0352 | −0.1951 | 0.0725 | C.ar | 0.1489 |
| C2 | 2.0834 | −0.0915 | 0.6474 | C.3 | 0.1387 |
| C3 | 2.3288 | 1.3986 | 0.5409 | C.2 | 0.1298 |
| C4 | 2.7343 | 2.1393 | 1.7840 | C.3 | 0.0249 |
| C5 | 1.6213 | 1.9380 | 2.8395 | C.3 | −0.0154 |
| C6 | 1.5391 | 0.4338 | 3.2099 | C.3 | 0.0664 |
| C7 | 1.2934 | −0.4401 | 1.9514 | C.3 | 0.0294 |
| C8 | 0.3791 | 0.1181 | 4.2040 | C.3 | 0.0429 |
| C9 | −1.0383 | 0.5073 | 3.6641 | C.3 | 0.0052 |
| C10 | −1.2030 | 0.2284 | 2.1659 | C.ar | −0.0334 |
| C11 | −0.0782 | −0.1163 | 1.4337 | C.ar | −0.0151 |
| C12 | −2.4171 | 0.3074 | 1.4505 | C.ar | −0.0499 |
| C13 | −2.4130 | 0.2019 | 0.0328 | C.ar | −0.0203 |
| C14 | −1.2074 | 0.0000 | −0.6793 | C.ar | 0.1404 |
| O15 | 1.2170 | −0.4755 | −0.4637 | O.3 | −0.2867 |
| C16 | 1.3253 | −1.9545 | 2.2801 | C.3 | −0.0592 |
| N17 | 0.4895 | −1.3246 | 4.5611 | N.3 | −0.2960 |
| C18 | 0.3363 | −2.2765 | 3.4315 | C.3 | −0.0091 |
| O19 | 2.8028 | 0.1380 | 3.8337 | O.3 | −0.3969 |
| O20 | −1.1968 | 0.0000 | −2.0760 | O.3 | 0.3351 |
| O21 | 2.1919 | 2.0008 | −0.5126 | O.2 | −0.3894 |
| C22 | −0.1632 | −1.7771 | 5.8169 | C.3 | 0.0022 |
| C23 | 0.2667 | −0.9142 | 7.0296 | C.3 | −0.0282 |
| C24 | −0.5945 | −1.0908 | 8.2998 | C.3 | −0.0488 |
| C25 | −0.7018 | 0.2063 | 7.4700 | C.3 | −0.0488 |
| H26 | −3.3439 | 0.2757 | −0.5190 | H | 0.0719 |
| H27 | −3.3515 | 0.4481 | 1.9839 | H | 0.0519 |
| H28 | −0.7033 | −2.2458 | 3.0686 | H | 0.0417 |
| H29 | 0.5379 | −3.3100 | 3.7583 | H | 0.0417 |
| H30 | 1.0537 | −2.5464 | 1.3901 | H | 0.0165 |
| H31 | 2.3491 | −2.2448 | 2.5610 | H | 0.0165 |
| H32 | 3.7066 | 1.7640 | 2.1382 | H | 0.0495 |
| H33 | 2.8430 | 3.2119 | 1.5551 | H | 0.0495 |
| H34 | 0.6739 | 2.3152 | 2.4251 | H | 0.0308 |
| H35 | 1.8585 | 2.5217 | 3.7437 | H | 0.0308 |
| H36 | −1.2074 | 1.5867 | 3.7999 | H | 0.0488 |
| H37 | −1.8236 | −0.0234 | 4.2195 | H | 0.0488 |
| H38 | 3.0581 | −0.5987 | 0.5948 | H | 0.0780 |
| H39 | 0.5866 | 0.7227 | 5.1003 | H | 0.0510 |
| H40 | −0.3069 | 0.0000 | −2.4176 | H | 0.2424 |
| H41 | 2.8163 | −0.7158 | 4.2555 | H | 0.2089 |
| H42 | 0.1871 | −2.7925 | 6.0602 | H | 0.0429 |
| H43 | −1.2569 | −1.8218 | 5.7021 | H | 0.0429 |
| H44 | 1.3391 | −0.7446 | 7.2194 | H | 0.0313 |
| H45 | −1.6257 | 0.3467 | 6.8884 | H | 0.0268 |
| H46 | −0.2477 | 1.1098 | 7.9059 | H | 0.0268 |
| H47 | −1.4559 | −1.7752 | 8.2529 | H | 0.0268 |
| H48 | −0.0805 | −1.0045 | 9.2699 | H | 0.0268 |

Through the use of these coordinates a pharmacophore may be defined by: (1) a hydrogen bonding moiety at a three-dimensional location corresponding to the hydroxyl at position 3 of naltrexone; (2) a hydrogen bonding moiety at a three-dimensional location corresponding to the hydroxyl at position 14 of naltrexone; (3) a hydrophobic moiety at a three-dimensional location corresponding to the cyclopropyl moiety appended to the nitrogen of naltrexone; and (4) a region of electron density at a three-dimensional location corresponding to the ethylene moiety at 6-position of naltrexone.

All publications and patent applications mentioned in this specification are herein incorporated by reference to the same extent as if each individual publication of patent application was specifically and individually indicated to be incorporated by reference. U.S. patent application Ser. Nos. 10/000,113 (filed Oct. 30, 2001-docket no. 12971US04); 10/000,107 (filed (Oct. 30, 2001-docket no. 13726US01); ______ (filed May 30, 2002-docket no. 13726US02) are each herein incorporated by reference.

The invention now being fully described, it will be apparent to one of ordinary skill in the art that many changes and modifications can be made thereto without departing from the spirit or scope of the appended claims.

What is claimed is:

1. A method of increasing efficacy of an anti-tumor agent comprising co-administering to a subject suffering from a multidrug resistant cancer:
(a) a dose of the anti-tumor agent, wherein the anti-tumor agent is a substrate of an ABC drug transporter, and
(b) a dose of an opioid inhibitor of the ABC drug transporter.

2. The method of claim 1, wherein the dose of the opioid inhibitor of the ABC drug transporter is sufficient to reduce efflux of the anti-tumor agent from a cancer cell, increase intracellular concentration of the anti-tumor agent in a cancer cell, or inhibit a host drug transporter.

3. The method of claim 1, wherein the anti-tumor agent is an Alkylating Agent, Antimetabolite, Vinca alkaloid, taxane, epipodophyllotoxin, Anthracycline, Antiproliferative agent, Tubulin Binding agent, Enediyne, anthracededione, substituted urea, methylhydrazine derivative, a member of the Pteridine family of drugs, Taxane, Dolastatin, Topoiosomerase inhibitor, Mytansinoid, or Platinum coordination complex.

4. The method of claim 1, wherein the dose of anti-tumor agent is a sub-therapeutic dose.

5. The method of claim 1, wherein the opioid inhibitor of the ABC drug transporter is a compound of the formula: 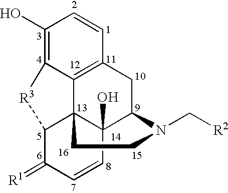
wherein R¹ is CH₂ or O;
wherein R² is a cycloalkyl, unsubstituted aromatic, alkyl or alkenyl; and
wherein R³ is O, CH₂ or NH.

6. The method of claim 1, wherein the opioid inhibitor of the ABC drug transporter is nalmefene, naltrexone or naloxone.

7. The method of claim 1, wherein the opioid inhibitor of the ABC drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

8. A method of increasing efficacy of an anti-tumor agent comprising co-administering to a subject having a cancer:
(a) a dose of the anti-tumor agent, wherein the anti-tumor agent is a substrate of an ABC drug transporter, and
(b) a dose of an opioid inhibitor of the ABC drug transporter.

9. The method of claim 8, wherein the dose of the opioid inhibitor of the ABC drug transporter is sufficient to increase the intracellular concentration of the anti-tumor agent in a cancer cell, or inhibit a host drug transporter.

10. The method of claim 8, wherein the dose of the anti-tumor agent is a sub-therapeutic dose.

11. The method of claim 8, wherein the anti-tumor agent is an Alkylating Agent, Antimetabolite, Vinca alkaloid, taxane, epipodophyllotoxin, Anthracycline, Antiproliferative agent, Tubulin Binding agent, Enediyne, anthracededione, substituted urea, methylhydrazine derivative, a member of the Pteridine family of drugs, Taxane, Dolastatin, Topoiosomerase inhibitor, Mytansinoid, or Platinum coordination complex.

12. The method of claim 8, wherein the opioid inhibitor of the ABC drug transporter is a compound of the formula: 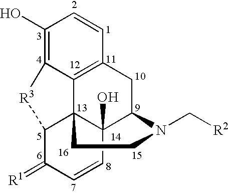
wherein R¹ is CH₂ or O;
wherein R² is a cycloalkyl, unsubstituted aromatic, alkyl or alkenyl; and
wherein R³is O, CH₂ or NH.

13. The method of claim 8, wherein the opioid inhibitor of the ABC drug transporter is nalmefene, naltrexone or naloxone.

14. The method of claim 8, wherein the opioid inhibitor of the ABC drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

15. A method of decreasing toxicity associated with treating a cancer patient with an anti-tumor agent comprising co-administering to a patient having a cancer:
(a) a dose of the anti-tumor agent, wherein the anti-tumor agent is a substrate of an ABC drug transporter, and
(b) a dose of an opioid inhibitor of the ABC drug transporter.

16. The method of claim 15, wherein the dose of the opioid inhibitor of the ABC drug transporter is sufficient to reduce efflux of the anti-tumor agent from a cancer cell, increase intracellular concentration of the anti-tumor agent in a cancer cell, or inhibit a host drug transporter.

17. The method of claim 15, wherein the dose of anti-tumor agent is a sub-therapeutic dose.

18. The method of claim 15, wherein the anti-tumor agent is an Alkylating Agent, Antimetabolite, Vinca alkaloid, taxane, epipodophyllotoxin, Anthracycline, Antiproliferative agent, Tubulin Binding agent, Enediyne, anthracededione, substituted urea, methylhydrazine derivative, a member of the Pteridine family of drugs, Taxane, Dolastatin, Topoiosomerase inhibitor, Mytansinoid, or Platinum coordination complex.

19. The method of claim 15, wherein the opioid inhibitor of the ABC drug transporter is a compound of the formula: 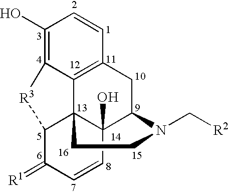
wherein R¹ is CH₂ or O;
wherein R² is a cycloalkyl, unsubstituted aromatic, alkyl or alkenyl; and
wherein R³ is O, CH₂ or NH.

20. The method of claim 15, wherein the opioid inhibitor of the ABC drug transporter is nalmefene, naltrexone or naloxone.

21. The method of claim 15, wherein the opioid inhibitor of the ABC drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

22. A method of decreasing toxicity associated with treating a cancer patient with an anti-tumor agent comprising administering to a patient having a cancer:
(a) a sub-therapeutic dose of the anti-tumor agent, wherein the anti-tumor agent is a substrate of an ABC drug transporter, and
(b) a dose of an opioid inhibitor of the ABC drug transporter.

23. The method of claim 22, wherein the dose of the opioid inhibitor of the ABC drug transporter is sufficient to increase the intracellular concentration of the anti-tumor agent in a cancer cell or inhibit a host drug transporter.

24. The method of claim 22, wherein the anti-tumor agent is an Alkylating Agent, Antimetabolite, Vinca alkaloid, taxane, epipodophyllotoxin, Anthracycline, Antiproliferative agent, Tubulin Binding agent, Enediyne, anthracededione, substituted urea, methylhydrazine derivative, a member of the Pteridine family of drugs, Taxane, Dolastatin, Topoiosomerase inhibitor, Mytansinoid, or Platinum coordination complex.

25. The method of claim 22, wherein the opioid receptor antagonist is a compound of the formula: 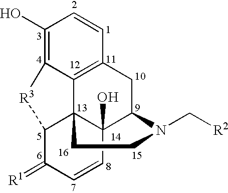
wherein R¹ is CH₂ or O;
wherein R² is a cycloalkyl, unsubstituted aromatic, alkyl or alkenyl; and
wherein R³ is O, CH₂ or NH.

26. The method of claim 22, wherein the opioid inhibitor of the ABC drug transporter is nalmefene, naltrexone or naloxone.

27. The method of claim 22, wherein the opioid inhibitor of the drug transporter is nalmefene.

28. The method of claim 22, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

29. A composition for treating multidrug resistant cancer cells comprising:
(a) an anti-tumor agent, wherein the anti-tumor agent is a substrate of an ABC drug transporter protein; and
(b) an opioid inhibitor of the ABC transporter protein.

30. The composition of claim 28, wherein the opioid receptor antagonist is a compound of the formula: 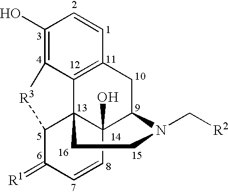
wherein R¹ is CH₂ or O;
wherein R² is a cycloalkyl, unsubstituted aromatic, alkyl or alkenyl; and
wherein R³ is O, CH₂ or NH.

31. The composition of claim 28, wherein the opioid inhibitor of the ABC drug transporter is nalmefene, naltrexone or naloxone.

32. The composition of claim 28, wherein the opioid inhibitor of the drug transporter is nalmefene.

33. The composition of claim 28, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

34. The composition of claim 28, wherein the anti-tumor agent is an Alkylating Agent, Antimetabolite, Vinca alkaloid, taxane, epipodophyllotoxin, Anthracycline, Antiproliferative agent, Tubulin Binding agent, Enediyne, anthracededione, substituted urea, methylhydrazine derivative, a member of the Pteridine family of drugs, Taxane, Dolastatin, Topoiosomerase inhibitor, Mytansinoid, or Platinum coordination complex.

35. A method of enhancing the anti-tumor activity of an anti-tumor agent against a multidrug resistant cancer cell comprising:
contacting the cancer cell with the anti-tumor agent and an opioid inhibitor of an ABC drug transporter in an amount effective to inhibit a drug transporter in the cancer cell.

36. The method of claim 35, wherein the opioid receptor antagonist is a compound of the formula: 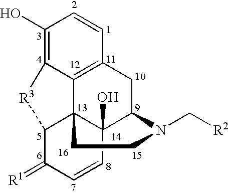
wherein R¹ is CH₂ or O;
wherein R² is a cycloalkyl, unsubstituted aromatic, alkyl or alkenyl; and
wherein R³ is O, CH₂ or NH.

37. The method of claim 35 wherein the opioid inhibitor of the ABC drug transporter is nalmefene, naltrexone or naloxone.

38. The method of claim 35, wherein the opioid inhibitor of the drug transporter is nalmefene.

39. The method of claim 35, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

40. The method of claim 35, wherein the anti-tumor agent is an Alkylating Agent, Antimetabolite, Vinca alkaloid, taxane, epipodophyllotoxin, Anthracycline, Antiproliferative agent, Tubulin Binding agent, Enediyne, anthracededione, substituted urea, methylhydrazine derivative, a member of the Pteridine family of drugs, Taxane, Dolastatin, Topoiosomerase inhibitor, Mytansinoid, or Platinum coordination complex.

41. A method of suppressing growth of a multidrug resistant cancer cell comprising:
contacting the cancer cell with a therapeutic or sub-therapeutic amount of an anti-tumor agent in the presence of an opioid inhibitor of an ABC drug transporter.

42. The method of claim 41, wherein the opioid inhibitor of the drug transporter is a compound of the formula: 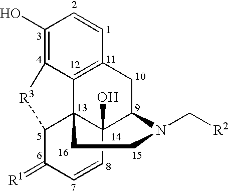
wherein R¹ is CH₂ or O;
wherein R² is a cycloalkyl, unsubstituted aromatic, alkyl or alkenyl; and
wherein R³ is O, CH₂ or NH.

43. The method of claim 41 wherein the opioid inhibitor of the ABC drug transporter is nalmefene, naltrexone or naloxone.

44. The method of claim 41, wherein the opioid inhibitor of the drug transporter is nalmefene.

45. The method of claim 41, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

46. The method of claim 41, wherein the anti-tumor agent is an Alkylating Agent, Antimetabolite, Vinca alkaloid, taxane, epipodophyllotoxin, Anthracycline, Antiproliferative agent, Tubulin Binding agent, Enediyne, anthracededione, substituted urea, methylhydrazine derivative, a member of the Pteridine family of drugs, Taxane, Dolastatin, Topoiosomerase inhibitor, Mytansinoid, or Platinum coordination complex.

47. A method of inhibiting a P-glycoprotein in a subject suffering from cancer comprising administering to the subject a P-glycoprotein inhibiting amount of an inhibitor of an ABC drug transporter, wherein the inhibitor is nalmefene, naltrexone or naloxone, wherein the inhibitor is administered before, with, or after the administration to the patient of a therapeutic or sub-therapeutic amount of an anti-tumor agent.

48. The method of claim 47, wherein the P-glycoprotein is PGP1a.

49. The method of claim 47, wherein the anti-tumor agent is an Alkylating Agent, Antimetabolite, Vinca alkaloid, taxane, epipodophyllotoxin, Anthracycline, Antiproliferative agent, Tubulin Binding agent, Enediyne, anthracededione, substituted urea, methylhydrazine derivative, a member of the Pteridine family of drugs, Taxane, Dolastatin, Topoiosomerase inhibitor, Mytansinoid, or Platinum coordination complex.

50. A method of inhibiting a P-glycoprotein in a subject suffering from cancer comprising administering to the subject P-glycoprotein inhibiting amount of an inhibitor of an ABC drug transporter, wherein the inhibitor of the ABC drug transporter is a compound of the formula: 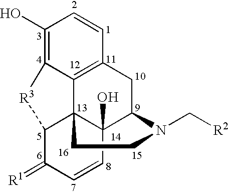
wherein R¹ is CH₂ or O;
wherein R² is a cycloalkyl, unsubstituted aromatic, alkyl or alkenyl; and
wherein R³ is O, CH₂ or NH,
wherein the inhibitor is administered before, with, or after the administration to the patient of a therapeutic or sub-therapeutic amount of an anti-tumor agent.

51. The method of claim 50, wherein the P-glycoprotein is PGP1a.

52. The method of claim 50, wherein the anti-tumor agent is an Alkylating Agent, Antimetabolite, Vinca alkaloid, taxane, epipodophyllotoxin, Anthracycline, Antiproliferative agent, Tubulin Binding agent, Enediyne, anthracededione, substituted urea, methylhydrazine derivative, a member of the Pteridine family of drugs, Taxane, Dolastatin, Topoiosomerase inhibitor, Mytansinoid, or Platinum coordination complex.

53. A method of identifying a compound for improved treatment of multidrug resistant cancers comprising:
(a) identifying an anti-tumor agent;
(b) assaying the ability of the anti-tumor agent to be transported across a membrane by an ABC protein; and
(c) repeating the transport assay to determine whether addition of an opioid receptor antagonist inhibits transport of the anti-tumor agent across the membrane,
whereby the compound which is active in the brain, is transported by an ABC protein and whose ABC protein-mediated transport is inhibited by the opioid receptor antagonist is identified.

54. The method of claim 53, wherein the opioid receptor antagonist is nalmefene, naltrexone or naloxone.

55. A method of enhancing the potency of a compound identified by the method of claim 53 comprising:
co-administering a therapeutic amount of the compound and an amount of an opioid receptor antagonist capable of inhibiting a drug transporter, wherein the amount of the opioid receptor antagonist is sufficient to reduce transport of the compound across a biological membrane.

56. A method for screening for an opioid inhibitor of an ABC drug transporter, comprising determining whether a potential opioid inhibitor inhibits growth of a cancer cell in the presence of sub-therapeutic amount of anti-tumor agent,
wherein the cancer cell expresses an ABC drug transporter, and
wherein said determining comprises comparing the growth of the cancer cell which expresses the ABC drug transporter, with growth of a second cancer cell which does not produce the ABC drug transporter, wherein the first and second cancer cells are grown in the presence of the sub-therapeutic amount of the anti-tumor agent.

57. A method for screening for an opioid inhibitor of an ABC drug transporter, comprising:
contacting a potential opioid inhibitor of an ABC drug transporter protein with the ABC drug transporter protein in the presence of a compound that is nalmefene, naltrexone or naloxone, wherein the compound is detectably labeled;
measuring the amount of detectably labeled compound bound to the ABC drug transporter; and
comparing the measured amount to the amount of detectably labeled compound bound by the ABC drug transporter when the drug transporter is contacted with the compound alone,
whereby a measured amount lower than the amount of compound bound to the ABC drug transporter when contacted alone identifies an opioid inhibitor of the ABC drug transporter.

58. The method of claim 57, wherein the potential opioid inhibitor of the ABC drug transporter is nalmefene.

59. A method of treating a cancer in an animal, comprising administering to the animal suffering from the cancer an anti-tumor agent and an ABC drug transporter inhibitor in an amount sufficient to increase the intracellular concentration of the anti-tumor agent in a cancer cell,
wherein the ABC drug transporter inhibitor increases the susceptibility of the cancer to the anti-tumor agent, and
wherein the ABC drug transporter inhibitor is nalmefene, naltrexone or naloxone.

60. A method of treating a cancer in an animal, comprising administering to the animal suffering from the cancer an anti-tumor agent and an ABC drug transporter inhibitor in an amount sufficient to increase the intracellular concentration of the anti-tumor agent in a cancer cell,
wherein the ABC drug transporter inhibitor increases the susceptibility of the cancer cell to the anti-tumor agent, and
wherein the ABC drug transporter inhibitor is a compound of the formula: 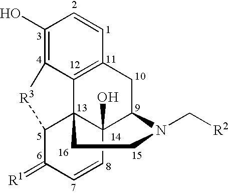
wherein R¹ is CH₂ or O;
wherein R² is a cycloalkyl, unsubstituted aromatic, alkyl or alkenyl; and
wherein R³ is O, CH₂ or NH.

61. A method of decreasing the toxicity of an anti-tumor agent comprising co-administering:
(a) a therapeutic or sub-therapeutic dose of the anti-tumor agent, wherein the anti-tumor agent is a substrate of an ABC drug transporter, and
(b) a dose of an opioid inhibitor of the ABC drug transporter.

62. A method of enhancing the potency of an anti-tumor agent comprising co-administering:
(a) a therapeutic or sub-therapeutic dose of the anti-tumor agent, wherein the anti-tumor agent is a substrate of an ABC drug transporter, and
(b) a dose of an opioid inhibitor of the ABC drug transporter.

63. A method of increasing efficacy of an anti-tumor agent comprising co-administering to a subject suffering from a multidrug resistant cancer:
(a) a dose of the anti-tumor agent, wherein the anti-tumor agent is a substrate of an ABC drug transporter, and
(b) a dose of an opioid inhibitor of the ABC drug transporter, wherein the dose of the opioid inhibitor of the ABC drug transporter is sufficient to reduce efflux of the anti-tumor agent from a cancer cell, and wherein the co-administration of the anti-tumor agent and the inhibitor is sufficient to inhibit the growth of the cancer.

64. A method of increasing efficacy of an anti-tumor agent comprising co-administering to a subject having a cancer:
(a) a dose of the anti-tumor agent, wherein the anti-tumor agent is a substrate of an ABC drug transporter, and
(b) a dose of an opioid inhibitor of the ABC drug transporter,
wherein the dose of the opioid inhibitor of the ABC drug transporter is sufficient to increase the intracellular concentration of the anti-tumor agent in a cancer cell, and wherein the co-administration of the anti-tumor agent and the inhibitor is sufficient to inhibit the growth of the cancer.

65. A method of decreasing toxicity associated with treating a cancer patient with an anti-tumor agent comprising co-administering to a patient having a cancer:
(a) a dose of the anti-tumor agent, wherein the anti-tumor agent is a substrate of an ABC drug transporter, and
(b) a dose of an opioid inhibitor of the ABC drug transporter,
wherein the dose of the opioid inhibitor of the ABC drug transporter is sufficient to reduce efflux of the anti-tumor agent from a cancer cell, and wherein the co-administration of the anti-tumor agent and the inhibitor is sufficient to inhibit the growth of the cancer.

66. A method of decreasing toxicity associated with treating a cancer patient with an anti-tumor agent comprising administering to a patient having a cancer:
(a) a sub-therapeutic dose of the anti-tumor agent, wherein the anti-tumor agent is a substrate of an ABC drug transporter, and
(b) a dose of an opioid inhibitor of the ABC drug transporter, wherein the dose of the opioid inhibitor of the ABC drug transporter is sufficient to increase the intracellular concentration of the anti-tumor agent in a cancer cell, and
wherein the co-administration of the anti-tumor agent and the inhibitor is sufficient to inhibit the growth of the cancer.


SEQUENCE LISTING (1 sequence)

SEQ ID NO: 1 - protein, 5 aa, Homo sapiens
LSGGQ